(19) United States
(12) Patent Application Publication
(10) Pub. No.: US 2026/0055011 A1
(43) Pub. Date: Feb. 26, 2026

(54) AMINE-BASED SOLVENTS FOR TEMPERATURE SWING SOLVENT EXTRACTION

(71) Applicant: The Board of Trustees of The University of Alabama, Tuscaloosa, AL (US)

(72) Inventors: Steven Thomas Weinman, Tuscaloosa, AL (US); Jason E. Bara, Tuscaloosa, AL (US); Heath Turner, Tuscaloosa, AL (US); Gabriel D. Barbosa, Tuscaloosa, AL (US)

(21) Appl. No.: 19/308,072

(22) Filed: Aug. 22, 2025

Related U.S. Application Data
(60) Provisional application No. 63/685,801, filed on Aug. 22, 2024.

(51) Int. Cl. C02F 1/26; B01D 11/04; C07C 215/10; C07C 217/04

(52) U.S. Cl. CPC C02F 1/265; B01D 11/0492; C07C 215/10; C07C 217/04; C02F 2303/16

(57) ABSTRACT

Disclosed are systems and methods for desalinating high-salinity brines via temperature swing solvent extraction (TSSE) using an amine-based solvent.

### CROSS-REFERENCE TO RELATED APPLICATIONS

This Application claims priority to and benefit under 35 U.S.C. § 119(e) of U.S. Provisional Patent Application Ser. No. 63/685,801, entitled “AMINE-BASED SOLVENTS FOR TEMPERATURE SWING SOLVENT EXTRACTION,” filed Aug. 22, 2024, which is hereby incorporated by reference in its entirety.

### STATEMENT OF GOVERNMENT SUPPORT

This invention was made with government support under Grant No. R23AC00435 awarded by the U.S. Department of the Interior, Bureau of Reclamation. The government has certain rights in the invention.

### BACKGROUND

Global factors such as population growth and climate change are causing severe water stress. This water stress affects many areas of life, including basic cleanliness, food production, energy generation, and materials manufacturing. To offset this growing demand, freshwater can be supplemented using nontraditional water sources such as those containing high total dissolved solids (TDS). One such method is to use reverse osmosis (RO) to desalinate seawater and inland brackish water. However, conventional RO can only treat water with TDS levels <70,000 ppm, creating large amounts of residual brine that must be disposed of responsibly. Brine transport and permitting for discharge is expensive and environmentally harmful. While some disposal options exist, such as brine concentrators and crystallizers, these options are expensive and energy intensive due to the evaporative phase change required. Thus, the reduction/elimination of brine presents an ongoing barrier for environmentally sustainable and economically feasible desalination processes.

Thermal distillation is the dominant approach to recover water from hypersaline brines. However, thermal evaporation techniques suffer from low efficiency, scaling and corrosion, and operating costs. Other membrane technologies, including high-pressure RO, forward osmosis, membrane distillation, and electrodialysis have been proposed to treat hypersaline brines. Unfortunately, these techniques are still likely to suffer from fouling/scaling, low productivity, and costly operation.

Temperature swing solvent extraction (TSSE) (also known as solvent extraction desalination) is a radically different water desalination technology that is membrane-less and avoids the thermodynamic constraints of evaporative processes. However, solvent-based desalination is still in its infancy and much research needs to be done to design a practical treatment system. TSSE is a desalination technique that requires the use of an organic solvent whose miscibility with water is strongly dependent on temperature. The long chain carboxylic acid or hydrophobic amine solvent dissolves water at one temperature to achieve brine concentration or crystallization, the phases are separated, and the water-in-solvent mixture is heated or cooled (depending on the solvent used) to cause the desalinated water to phase separate. However, the solvents studied for TSSE suffer from a few issues. One, the best performing solvent, N,N-diisopropylamine (DIPA), dissolves at a non-negligible level (≥20,000 ppm) in water, requiring the use of RO to polish the final product water. Additionally, DIPA has a relatively low boiling point of 84° C., which increases the risk for severe evaporative solvent loss.

TSSE is a promising technique for brine concentration (and crystallization). However, for this technique to become commercially viable, new cost-effective solvents need to be developed which have a higher capacity for water and exhibit a lower water solubility. The methods and compositions disclosed herein address these and other needs.

### SUMMARY

Disclosed herein are compounds, compositions, methods for making and using such compounds and compositions. In various aspects disclosed herein is a method for desalinating high-salinity brines. In some aspects, the method includes contacting a feed brine with an amine-based solvent under conditions effective for temperature swing solvent extraction (TSSE) to produce a substantially desalinated product, wherein the amine-based solvent comprises sec-butyl-isopropyl-amine and/or a glycerol-based amine solvent.

In some aspects, the amine-based solvent comprises sec-butyl-isopropyl-amine represented by:

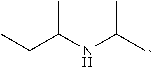

or a salt thereof.

In some aspects, the glycerol-based amine solvent is represented by Formula I, or a salt thereof:

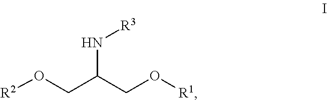

wherein each R¹, R², and R³ is independently hydrogen, a substituted or unsubstituted linear, branch, or cyclic alkyl, a substituted or unsubstituted linear, branch, or cyclic alkoxy, a substituted or unsubstituted linear, branch, or cyclic alkoxyalkyl, or a substituted or unsubstituted linear, branch, or cyclic alkenyl; with the proviso that at least one of R¹, R², and R³ is not hydrogen.

In some aspects, each R¹, R², and R³ is independently hydrogen, a substituted or unsubstituted linear, branch, or cyclic C₁-C₆ alkyl, a substituted or unsubstituted linear, branch, or cyclic C₁-C₆ alkoxy, a substituted or unsubstituted linear, branch, or cyclic C₁-C₆ alkoxyalkyl, or a substituted or unsubstituted linear, branch, or cyclic C₁-C₆ alkenyl; with the proviso that at least one of R¹, R², and R³ is not hydrogen.

In some aspects, each R¹, R², and R³ is independently hydrogen, a substituted or unsubstituted linear, branch, or cyclic C₁-C₃ alkyl, a substituted or unsubstituted linear, branch, or cyclic C₁-C₃ alkoxy, a substituted or unsubstituted linear, branch, or cyclic C₁-C₃ alkoxyalkyl, or a substituted or unsubstituted linear, branch, or cyclic C₁-C₆ alkenyl; with the proviso that at least one of R¹, R², and R³ is not hydrogen.

In some aspects, according to Formula I, each of R¹, R², and R³ are the same. In some aspects according to Formula I, R¹ and R² are the same. In some aspects according to Formula I, at least one of R¹, R², and R³ is different. In some aspects according to Formula I, R¹, R², and R³ are all different. In some aspects, none of R¹, R², and R³ are hydrogen.

In some aspects, the glycerol-based amine solvent is represented by Formula II, or a salt thereof:

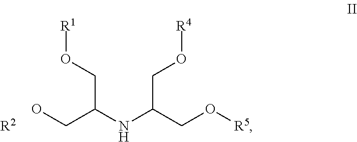

wherein each R¹, R², R⁴, and R⁵ is independently hydrogen, a substituted or unsubstituted linear, branch, or cyclic alkyl, a substituted or unsubstituted linear, branch, or cyclic alkoxy, a substituted or unsubstituted linear, branch, or cyclic alkoxyalkyl, or a substituted or unsubstituted linear, branch, or cyclic alkenyl; with the proviso that at least one of R¹, R², R⁴, and R⁵ is not hydrogen.

In some aspects, each R¹, R², R⁴, and R⁵ is independently hydrogen, a substituted or unsubstituted linear, branch, or cyclic C₁-C₆ alkyl, a substituted or unsubstituted linear, branch, or cyclic C₁-C₆ alkoxy, a substituted or unsubstituted linear, branch, or cyclic C₁-C₆ alkoxyalkyl, or a substituted or unsubstituted linear, branch, or cyclic C₁-C₆ alkenyl; with the proviso that at least one of R¹, R², R⁴, and R⁵ is not hydrogen.

In some aspects, each R¹, R², R⁴, and R⁵ is independently hydrogen, a substituted or unsubstituted linear, branch, or cyclic C₁-C₃ alkyl, a substituted or unsubstituted linear, branch, or cyclic C₁-C₃ alkoxy, a substituted or unsubstituted linear, branch, or cyclic C₁-C₃ alkoxyalkyl, or a substituted or unsubstituted linear, branch, or cyclic C₁-C₆ alkenyl; with the proviso that at least one of R¹, R², R⁴, and R⁵ is not hydrogen.

In some aspects, R¹, R², R⁴, and R⁵ are the same. In some aspects, at least one of R¹, R², R⁴, and R⁵ is different. In some aspects, at least two of R¹, R², R⁴, and R⁵ are different.

In some aspects, each of R¹, R², R⁴, and R⁵ is different. In some aspects, the amine-based solvent comprises a substantially symmetrical compound. In some aspects, none of R¹, R², R⁴, and R⁵ are hydrogen.

In some aspects, the amine-based solvent comprises more than one amine-based solvent.

In some aspects, the conditions effective for TSSE comprise:
contacting the feed brine with the amine-based solvent at a first temperature to form an extractant phase comprising water from the feed brine dissolved in the solvent and a raffinate;
separating the extractant phase and the raffinate;
heating or cooling the extractant phase to second temperature to thereby adjust the miscibility of the water in the amine-based solved to form the substantially desalinated water product and a solvent phase; and
separating the substantially desalinated water product from the solvent phase.

In some aspects, the method further includes recycling the separated solvent phase for additional TSSE processes.

In some aspects, the feed brine has a total dissolved solids of 50,000 ppm or more (e.g., 75,000 ppm or more, 100,000 ppm or more, 150,000 ppm or more, 200,000 ppm or more, or 250,000 ppm or more).

In another aspect, disclosed herein is a method for forming a solvent for a temperature swing solvent extraction (TSSE) process.

In another aspect, disclosed herein is a method for selecting a solvent for a TSSE process. In some aspects, the method includes providing a library of compounds for the TSSE process; determining one or more electrostatic properties for each of the compounds; and predicting one or more properties of the compounds as solvents in a TSSE process.

In various aspects, disclosed herein are systems configured to perform any one of the methods described herein.

Additional advantages of the disclosed subject matter will be set forth in part in the description that follows and the Figures, and in part will be obvious from the description, or can be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE FIGURES

The accompanying figures, which are incorporated in and constitute a part of this specification, illustrate several aspects described below.

FIG. 1 shows an example system for desalinating a high-salinity brine according to an exemplary aspect.

FIG. 2A shows initial configurations of (panel a) bulk DIPA-water mixture and (panel b) DIPA-water interfacial system.

FIG. 2B shows the average solvent properties within each cluster: (panel a) density, (panel b) solvation free energy of water

$$( {μ}_{w}^{∞} ) ,$$

(panel c) number of HBs per solvent molecule, and (panel d) nitrogen site partial charge. The properties are obtained by averaging among the solvents within each cluster, and the error bars are calculated assuming a confidence level of 0.95.

FIG. 3 shows the radial distribution function between the hydrogen site (H) bonded the carbon atom of DIPA and oxygen site (Ow) of the water molecule for different fixed point charge models and a polarizable one (AMOEBA).

FIG. 4 shows cluster members of (panel a) C1, (panel b) C2, (panel c) C3, and (panel d) C4 obtained using the k-means method and pure solvent as well as solvent-water properties as inputted features. Molecule labels are presented in Table 7.

FIG. 5 shows energy consumption versus total dissolved solids concentration for various desalination technologies.

FIG. 6 shows a general illustration of the working principles of temperature swing solvent extraction (TSSE).

FIG. 7 shows glycerol derived from natural sources and associated “activated” forms (top). Transformation to green chemical platforms (bottom) for brine desalination.

FIG. 8 shows electron density of glycerol mapped from the SCF density.

FIG. 9 shows automated identification of key interaction sites on prototype solvent molecules (left); automated classification of the solvent-solvent and solvent-water interactions (% contribution to overall interaction energy) among dozens of prototype solvent candidates (blue=electrostatic, red=dispersion, grey=repulsion) (right).

FIG. 10 shows a general overview of simulation screening and experimental workflow.

FIG. 11 shows PMF calculations used to evaluate transport barriers from the brine phase to the solvent phase.

### DETAILED DESCRIPTION

The materials, compounds, compositions, articles, and methods described herein may be understood more readily by reference to the following detailed description of specific aspects of the disclosed subject matter and the Examples and Figures included therein.

Before the present materials, compounds, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings: Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “the compound” includes mixtures of two or more such compounds, reference to “an agent” includes mixture of two or more such agents, and the like.

“Optional” or “optionally” means that the subsequently described event or circumstance can or cannot occur, and that the description includes instances where the event or circumstance occurs and instances where it does not.

“Phase,” as used herein, generally refers to a region of material having a substantially uniform composition which is a distinct and physically separate portion of a heterogeneous system. The term “phase” does not imply that the material making up a phase is a chemically pure substance, but merely that physical properties of the material making up the phase are essentially uniform throughout the material, and that these physical properties differ significantly from the physical properties of another phase within the material. Examples of physical properties include density, index of refraction, and chemical composition.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

### Chemical Definitions

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” as used herein refers to a non-aromatic hydrocarbon group and includes branched and unbranched, alkyl, alkenyl, or alkynyl groups.

The term “alkyl” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halides, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “alkoxy” as used herein is an alkyl group bound through a single, terminal ether linkage; that is, an “alkoxy” group can be defined as —OZ¹ where Z¹ is alkyl as defined above.

The term “alkenyl” as used herein is a hydrocarbon group of from 2 to 24 carbon atoms with a structural formula containing at least one carbon-carbon double bond. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “alkynyl” as used herein is a hydrocarbon group of 2 to 24 carbon atoms with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “aryl” as used herein is a group that contains any carbon-based aromatic group including, but not limited to, benzene, naphthalene, phenyl, biphenyl, phenoxybenzene, and the like. The term “heteroaryl” is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is included in the term “aryl,” defines a group that contains an aromatic group that does not contain a heteroatom. The aryl or heteroaryl group can be substituted or unsubstituted. The aryl or heteroaryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The term “aldehyde” as used herein is represented by the formula C(O)H. Throughout this specification “C(O)” or “CO” is a shorthand notation for C═O, which is also referred to herein as a “carbonyl.”

The terms “amine” or “amino” as used herein are represented by the formula NZ¹Z², where Z¹ and Z² can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above. “Amido” is

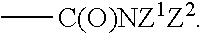

The term “carboxylic acid” as used herein is represented by the formula C(O)OH. A “carboxylate” or “carboxyl” group as used herein is represented by the formula

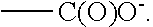

The term “carbamide” means compounds represented by the form —N(Z¹)—(CO)N(Z¹)₂ where each Z¹ can be, independently, an alkyl, alkenyl, alkynyl, aryl, arylalkyl, cycloalkyl, carbonyl, ether, haloalkyl, heteroaryl and heterocyclyl.

The term “carbamate” means compounds represented by the form —Z¹OC(O)N(Z¹)—, —Z¹OC(O)N(Z¹) Z¹—, or —OC(O)N(Z¹)₂, where each Z¹ can be, independently, an alkoxy, aryloxy, alkyl, alkenyl, alkynyl, aryl, arylalkyl, cycloalkyl, ether, formyl, haloalkyl, heteroaryl, and heterocyclyl. Carbamates include, e.g., arylcarbamates and heteroaryl carbamates.

The term “ester” as used herein is represented by the formula —OC(O)Z¹ or
—C(O)OZ¹, where Z¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula Z¹C(O)Z², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” or “halogen” as used herein refers to the fluorine, chlorine, bromine, and iodine.

The term “hydroxyl” as used herein is represented by the formula —OH.

The term “nitro” as used herein is represented by the formula —NO₂.

The term “silyl” as used herein is represented by the formula —SiZ¹Z²Z³, where Z¹, Z², and Z³ can be, independently, hydrogen, alkyl, halogenated alkyl, alkoxy, alkenyl, alkynyl, aryl, heteroaryl, cycloalkyl, cycloalkenyl, heterocycloalkyl, or heterocycloalkenyl group described above.

The term “sulfonyl” is used herein to refer to the sulfo-oxo group represented by the formula S(O)₂Z¹, where Z¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH—.

The term “thiol” as used herein is represented by the formula —SH.

The term “thio” as used herein is represented by the formula —S—.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

Reference will now be made in detail to specific aspects of the disclosed materials, compounds, compositions, articles, and methods, examples of which are illustrated in the accompanying Examples and Figures.

### Methods and Systems for Desalinating High-Salinity Brines

Disclosed herein are methods for desalinating high-salinity brines. In some aspects, the method includes contacting a feed brine with an amine-based solvent under conditions effective for temperature swing solvent extraction (TSSE) to produce a substantially desalinated product, wherein the amine-based solvent comprises sec-butyl-isopropyl-amine and/or a glycerol-based amine solvent.

In some aspects, the amine-based solvent comprises sec-butyl-isopropyl-amine (CAS 33546-49-5) represented by:

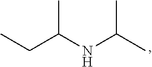

or a salt thereof. The solvent can comprise a single enantiomer of sec-butyl-isopropyl-amine, i.e., either the S or the R, enantiomer, or a racemic mixture of S and R, or a scalemic mixture of S and R enantiomers.

In some aspects, the amine-based solvent includes a glycerol-based amine solvent. The glycerol-based amine solvent may be a glycerol derivative. As used herein, a glycerol derivative refers to a compound that is derived from or includes a significant portion of glycerol as a backbone or core structural component (e.g., substituted with an amine group).

Glycerol, 1,2,3-trihydroxypropane, is a simple polyol comprising a propane with three alcohol functional groups. Glycerol has a wide range of applications, including use as an organic solvent and as a sweetener in foods. Glycerol is a byproduct of biodiesel production, and due to the recent expansion of biodiesel product, there is a glut of glycerol.

In some aspects, the glycerol-based amine solvent is represented by Formula I, or a salt thereof:

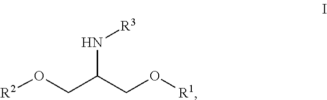

wherein each R¹, R², and R³ is independently hydrogen, a substituted or unsubstituted linear, branch, or cyclic alkyl, a substituted or unsubstituted linear, branch, or cyclic alkoxy, a substituted or unsubstituted linear, branch, or cyclic alkoxyalkyl, or a substituted or unsubstituted linear, branch, or cyclic alkenyl; with the proviso that at least one of R¹, R², and R³ is not hydrogen.

In some aspects according to Formula I, each R¹, R², and R³ is independently hydrogen, a substituted or unsubstituted linear, branch, or cyclic C₁-C₆ alkyl, a substituted or unsubstituted linear, branch, or cyclic C₁-C₆ alkoxy, a substituted or unsubstituted linear, branch, or cyclic C₁-C₆ alkoxyalkyl, or a substituted or unsubstituted linear, branch, or cyclic C₁-C₆ alkenyl; with the proviso that at least one of R¹, R², and R³ is not hydrogen.

In some aspects according to Formula I, each R¹, R², and R³ is independently hydrogen, a substituted or unsubstituted linear, branch, or cyclic C₁-C₃ alkyl, a substituted or unsubstituted linear, branch, or cyclic C₁-C₃ alkoxy, a substituted or unsubstituted linear, branch, or cyclic C₁-C₃ alkoxyalkyl, or a substituted or unsubstituted linear, branch, or cyclic C₁-C₆ alkenyl; with the proviso that at least one of R¹, R², and R³ is not hydrogen.

In some aspects, according to Formula I, each of R¹, R², and R³ are the same. In some aspects according to Formula I, R¹ and R² are the same. In some aspects according to Formula I, at least one of R¹, R², and R³ is different. In some aspects according to Formula I, R¹, R², and R³ are all different. In some aspects, none of R¹, R², and R³ are hydrogen.

In some aspects, the glycerol-based amine solvent is represented by Formula II, or a salt thereof:

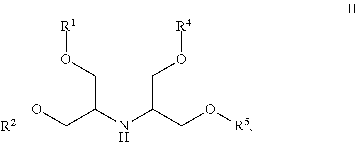

wherein each R¹, R², R⁴, and R⁵ is independently hydrogen, a substituted or unsubstituted linear, branch, or cyclic alkyl, a substituted or unsubstituted linear, branch, or cyclic alkoxy, a substituted or unsubstituted linear, branch, or cyclic alkoxyalkyl, or a substituted or unsubstituted linear, branch, or cyclic alkenyl; with the proviso that at least one of R¹, R², R⁴, and R⁵ is not hydrogen.

In some aspects according to Formula II, each R¹, R², R⁴, and R⁵ is independently hydrogen, a substituted or unsubstituted linear, branch, or cyclic C₁-C₆ alkyl, a substituted or unsubstituted linear, branch, or cyclic C₁-C₆ alkoxy, a substituted or unsubstituted linear, branch, or cyclic C₁-C₆ alkoxyalkyl, or a substituted or unsubstituted linear, branch, or cyclic C₁-C₆ alkenyl; with the proviso that at least one of R¹, R², R⁴, and R⁵ is not hydrogen.

In some aspects according to Formula II, each R¹, R², R⁴, and R⁵ is independently hydrogen, a substituted or unsubstituted linear, branch, or cyclic C₁-C₃ alkyl, a substituted or unsubstituted linear, branch, or cyclic C₁-C₃ alkoxy, a substituted or unsubstituted linear, branch, or cyclic C₁-C₃ alkoxyalkyl, or a substituted or unsubstituted linear, branch, or cyclic C₁-C₆ alkenyl; with the proviso that at least one of R¹, R², R⁴, and R⁵ is not hydrogen.

In some aspects, R¹, R², R⁴, and R⁵ are the same. In some aspects, at least one of R¹, R², R⁴, and R⁵ is different. In some aspects, at least two of R¹, R², R⁴, and R⁵ are different. In some aspects, each of R¹, R², R⁴, and R⁵ is different. In some aspects, the amine-based solvent comprises a substantially symmetrical compound. In some aspects, none of R¹, R², R⁴, and R⁵ are hydrogen.

In some aspects, according to any of Formulas I-II, the amine-based solvent comprises a substantially symmetric compound. In some aspects, the amine-based solvent comprises more than one amine-based solvent.

In some aspects, the conditions effective for TSSE includes:
contacting the feed brine with the amine-based solvent at a first temperature to form an extractant phase comprising water from the feed brine dissolved in the solvent and a raffinate;
separating the extractant phase and the raffinate (e.g., via gravity separation); heating or cooling the extractant phase to second temperature to thereby adjust the miscibility of the water in the amine-based solved to form the substantially desalinated water product and a solvent phase; and
separating the substantially desalinated water product from the solvent phase. In some aspects, the first temperature is higher than the second temperature. In some aspects, the first temperature is lower than the second temperature. In some aspects, the method further includes recycling the separated solvent phase for additional TSSE processes.

In some aspects, the feed brine has a total dissolved solids of 50,000 ppm or more (e.g., 75,000 ppm or more, 100,000 ppm or more, 150,000 ppm or more, 200,000 ppm or more, or 250,000 ppm or more).

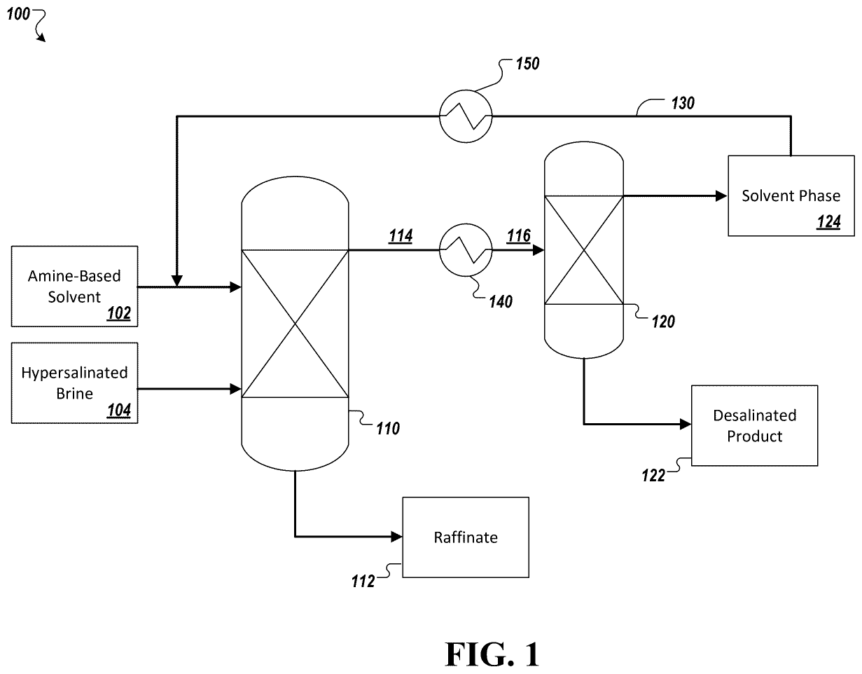

In various aspects, disclosed herein are systems configured to perform any one of the methods described herein. For example, FIG. 1 illustrates an example system 100 for desalinating high-salinity brines using temperature swing solvent extraction (TSSE) according to one aspect.

In the system 100, a feed brine 104 and an amine-based solvent 102 are contacted in an extraction vessel 110 (e.g., a liquid-liquid extraction column) to form an extractant phase 114 including a portion of water from the feed brine 104 dissolved in the amine-based solvent 102 and a raffinate 112. In various aspects, the amine-based solvent includes sec-butyl-isopropyl-amine and/or a glycerol-based amine solvent. In some aspects, the amine-based solvent is sec-butyl-isopropyl-amine. In some aspects, the amine-based solvent is glycerol-based amine solvent.

In some aspects, it is advantageous to heat and/or cool the feed brine 104 and/or amine-based solvent 102 prior to or during the extraction. For example, the feed brine 104 and/or amine-based solvent 102 can be adjusted to a first temperature to optimize the amount of water from the feed brine 104 dissolved in the amine-based solvent 102 during extraction. Determination of the first temperature typically depends on a number of factors, including, for example, the concentration of salts in the feed brine, the molecular characteristics of the amine-based solvent (e.g., polarity, size), and physical conditions (e.g., external pressure). In this regard, the optimal first temperature may be explored experimentally or can be estimated using molecular simulations.

In some aspects, the glycerol-based amine solvent is represented by Formula I, or a salt thereof:

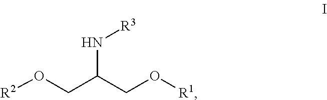

wherein each R¹, R², and R³ is independently hydrogen, a substituted or unsubstituted linear, branch, or cyclic alkyl, a substituted or unsubstituted linear, branch, or cyclic alkoxy, a substituted or unsubstituted linear, branch, or cyclic alkoxyalkyl, or a substituted or unsubstituted linear, branch, or cyclic alkenyl; with the proviso that at least one of R¹, R², and R³ is not hydrogen.

In some aspects according to Formula I, each R¹, R², and R³ is independently hydrogen, a substituted or unsubstituted linear, branch, or cyclic C₁-C₆ alkyl, a substituted or unsubstituted linear, branch, or cyclic C₁-C₆ alkoxy, a substituted or unsubstituted linear, branch, or cyclic C₁-C₆ alkoxyalkyl, or a substituted or unsubstituted linear, branch, or cyclic C₁-C₆ alkenyl; with the proviso that at least one of R¹, R², and R³ is not hydrogen.

In some aspects according to Formula I, each R¹, R², and R³ is independently hydrogen, a substituted or unsubstituted linear, branch, or cyclic C₁-C₃ alkyl, a substituted or unsubstituted linear, branch, or cyclic C₁-C₃ alkoxy, a substituted or unsubstituted linear, branch, or cyclic C₁-C₃ alkoxyalkyl, or a substituted or unsubstituted linear, branch, or cyclic C₁-C₆ alkenyl; with the proviso that at least one of R¹, R², and R³ is not hydrogen.

In some aspects, according to Formula I, each of R¹, R², and R³ are the same. In some aspects according to Formula I, R¹ and R² are the same. In some aspects according to Formula I, at least one of R¹, R², and R³ is different. In some aspects according to Formula I, R¹, R², and R³ are all different. In some aspects, none of R¹, R², and R³ are hydrogen.

In some aspects, the glycerol-based amine solvent is represented by Formula II, or a salt thereof:

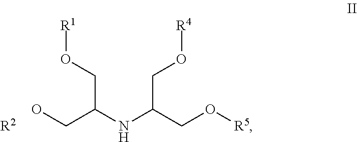

wherein each R¹, R², R⁴, and R⁵ is independently hydrogen, a substituted or unsubstituted linear, branch, or cyclic alkyl, a substituted or unsubstituted linear, branch, or cyclic alkoxy, a substituted or unsubstituted linear, branch, or cyclic alkoxyalkyl, or a substituted or unsubstituted linear, branch, or cyclic alkenyl; with the proviso that at least one of R¹, R², R⁴, and R⁵ is not hydrogen.

In some aspects according to Formula II, each R¹, R², R⁴, and R⁵ is independently hydrogen, a substituted or unsubstituted linear, branch, or cyclic C₁-C₆ alkyl, a substituted or unsubstituted linear, branch, or cyclic C₁-C₆ alkoxy, a substituted or unsubstituted linear, branch, or cyclic C₁-C₆ alkoxyalkyl, or a substituted or unsubstituted linear, branch, or cyclic C₁-C₆ alkenyl; with the proviso that at least one of R¹, R², R⁴, and R⁵ is not hydrogen.

In some aspects according to Formula II, each R¹, R², R⁴, and R⁵ is independently hydrogen, a substituted or unsubstituted linear, branch, or cyclic C₁-C₃ alkyl, a substituted or unsubstituted linear, branch, or cyclic C₁-C₃ alkoxy, a substituted or unsubstituted linear, branch, or cyclic C₁-C₃ alkoxyalkyl, or a substituted or unsubstituted linear, branch, or cyclic C₁-C₆ alkenyl; with the proviso that at least one of R¹, R², R⁴, and R⁵ is not hydrogen.

In some aspects, R¹, R², R⁴, and R⁵ are the same. In some aspects, at least one of R¹, R², R⁴, and R⁵ is different. In some aspects, at least two of R¹, R², R⁴, and R⁵ are different.

In some aspects, each of R¹, R², R⁴, and R⁵ is different. In some aspects, the amine-based solvent comprises a substantially symmetrical compound. In some aspects, none of R¹, R², R⁴, and R⁵ are hydrogen.

In some aspects, according to any of Formulas I-II, the amine-based solvent comprises a substantially symmetric compound. In some aspects, the amine-based solvent comprises more than one amine-based solvent.

Since some of the water in the feed brine 104 is dissolved in the amine-based solvent 102, the raffinate 112 includes a higher concentration of salts compared to the feed brine 104. The concentrated raffinate 112 may be subjected to additional (e.g., 1, 2, 3, 4, 5, 6, 7, 8, 9, 10) separation processes (e.g., further liquid-liquid extractions using amine-based solvents) or disposed. However, in some aspects, The additional separation processes may be arranged to maximize recovery of product water from the feed brine 104 without incurring substantial energy costs.

Following the separation of the extractant phase 114 and the raffinate 112, the extractant phase 114 is subjected to a temperature swing operation via heat exchanger 140. The temperature swing operation involves adjusting the temperature of the extractant 114 from a first temperature to a second temperature thereby affecting the miscibility of dissolved water in the amine-based solvent. In some aspects, the temperature swing is achieved by raising the temperature of the extractant 114. In some aspects, the temperature swing is achieved by lowering the temperature of the extractant 114. The temperature adjusted mixture 116 is then subjected to a separation vessel 120 (e.g., a liquid-liquid extraction column) to provide biphasic mixture including a solvent phase 124 and a desalinated water product 122. The solvent phase 124 and the desalinated water product 122 may be separated using any known technique, such as gravity separation. Although the heat exchanger 140 and separation vessel 120 are shown as separate elements in FIG. 1, it is also envisioned that the temperature swing operation via the heat exchanger 140 can occur within the separation vessel 120.

The desalinated water product 122 includes substantially purified water. As used herein, the term “substantially purified water” refers water that includes 80 wt. % or more of water, e.g., 82 wt. % or more, 84 wt. % or more, 86 wt. % or more, 88 wt. % or more, 90 wt. % or more, 92 wt. % or more, 94 wt. % or more, 95 wt. % or more, 96 wt. % or more, 97 wt. % or more, 98 wt. % or more, 99 wt. % or more, 99.5 wt. % or more, or 99.9 wt. % or more. In some aspects, an amount of total dissolved solids (TDS) in the feed brine is reduced by 80% or more compared to the desalinated product, e.g., 82% or more, 84% or more, 86% or more, 88% or more, 90% or more, 92% or more, 94% or more, 95% or more, 96% or more, 97% or more, 98% or more, 99% or more, 99.5% or more, or 99.9% or more. In some aspects, the system is configured to recover 30% or more of the water in the feed brine, e.g., 35% or more, 40% or more, 45% or more, 50% or more, 55% or more, 60% or more, 65% or more, 70% or more, 75% or more, 80% or more, 85% or more, 90% or more, 95% or more, 96% or more, 97% or more, 98% or more, 99% or more, 99.5% or more, or 99.9% or more. In some aspects, the desalinated product is subject to further processing (e.g., reverse osmosis). In some aspects, the desalinated product is recovered without any further processing or separation steps.

System 100 shown in FIG. 1 further includes a solvent phase recovery 130 in which the solvent phase 124 is recycled and returned for use in the desalination of feed brines. The solvent recovery 130 can include various conditioning processes to ensure the proper conditions for desalination are met. For example, the solvent phase recovery 130 includes a heat exchanger 150 which adjust the temperature of the solvent if needed before mixing with the feed brine 104 in the separation vessel 110. Although heat exchangers 140 and 150 are shown as separate elements in FIG. 1, in some aspects, the various streams may be thermally coupled such that heat from one location is used to drive a temperature increase at another location in the system.

### EXAMPLES

To further illustrate the principles of the present disclosure, the following examples are put forth so as to provide those of ordinary skill in the art with a complete disclosure and description of how the compositions, articles, and methods claimed herein are made and evaluated. They are intended to be purely exemplary of the invention and are not intended to limit the scope of what the inventors regard as their disclosure. Efforts have been made to ensure accuracy with respect to numbers (e.g., amounts, temperatures, etc.); however, some errors and deviations should be accounted for. Unless indicated otherwise, temperature is ° C. or is at ambient temperature, and pressure is at or near atmospheric. There are numerous variations and combinations of process conditions that can be used to optimize product quality and performance. Only reasonable and routine experimentation will be required to optimize such process conditions.

### Example 1: Characterization of Amine-Based Solvents

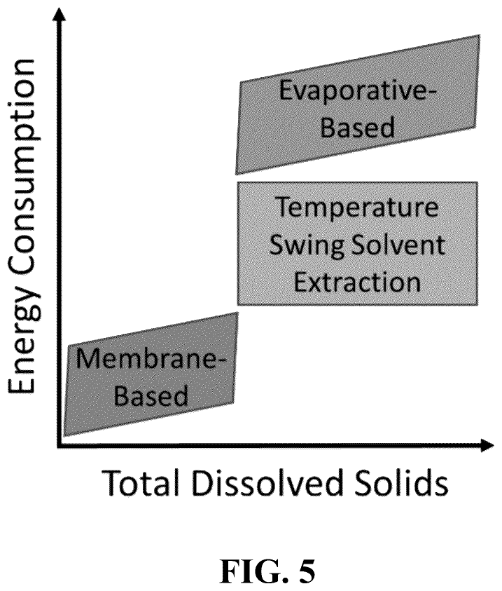

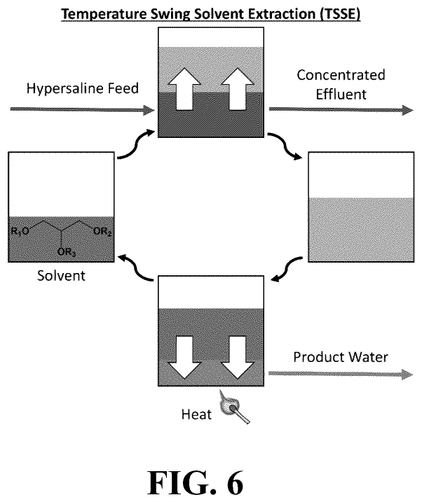

Introduction. Over the last few years, alternative methods to thermal and reverse osmosis techniques have been proposed.¹⁻⁴ Notably, temperature swing solvent extraction (TSSE) has proven effective for treating high-salinity brines. Temperature swing solvent extraction (TSSE) (also known as solvent extraction desalination) is a desalination technique that uses of an organic solvent whose miscibility with water is strongly dependent on temperature and can avoid the thermodynamic constraints of evaporative processes (FIGS. 5 and 6). Properly selecting a low-polarity water-selective organic solvent is the main issue in this approach. However, due to the several factors involved (solubility of salt in solvent phase, solvent toxicity, etc.), selecting an optimum solvent formulation can be challenging. Several different solvent classes have been evaluated for TSSE, including long-chain fatty acids,⁵⁻⁷ imidazolium-based ionic liquids (ILs),⁸ and amines⁷⁷. Short-chain secondary (2°) amine, such as diisopropylamine (DIPA, C₆H₁₅N), have demonstrated some of the best performance to date.⁹⁻¹⁴

Although some effort has been made to understand the TSSE performance of different amine-based solvents, the specific molecular features of solvent candidates are still an open issue. To investigate this issue, this study used molecular simulation techniques to better understand the molecular features and thermodynamic properties of water uptake in amine-based solvents. Initially, nearly 60 amine-based solvents were selected, restricted to molecular compositions containing from four to six carbons. The list of solvents, their chemical structures, and labels are shown in Table 7. For purposes of this study, these specific solvents were chosen as they represent 6-carbon analogues of the DIPA (M0) molecule in order to explore the influence of different molecular characteristics, such as branching, aliphaticity, and nitrogen location in the solvent structure.

Molecular Simulation Methodology. The MD simulations were carried out using the Gromacs package version 2021.1 (Lindahl, 2021), and the initial molecular configurations were built using the Packmol package. (Martinez, 2009). Based on the initial coordinates, the energy was minimized using the steepest decedent method. Then, the Parrinello-Rahman (Nos6, 1983; Parrinello, 1981) and v-rescaling method (Bussi, 2007) were used to simulate an isobaric-isothermic (NPT) ensemble, with time constants of 5 ps and 1 ps, respectively; the equations of motion were integrated using the leap-frog algorithm with a timestep of 2 fs.

The OPLS-AA force field bonded and non-bonded parameters were derived using the LigParGen web server. (Jorgensen, 2005; Dodda, 2017). Consistent with the OPLS-AA parameterization, partial atomic charges were calculated using the CHELPG method, (Breneman, 1990) based on the optimized molecular structures obtained via density functional theory (DFT) calculations performed at the B3LYP/6-31+(d,p) level of theory. All DFT calculations were carried out using Gaussian09. (Frisch, 2009). The semi-empirical 1.14*CM1A-LBCC charge method, (Jorgensen, 2005) known for its compatibility with OPLS-AA, was also tested to evaluate the influence of the charge method. As for water models, the SPC/E water model was used due to its good performance in predicting pure water properties (Vega, 2011) and its compatibility with the OPLS-AA force field. AmberTools 22 was used to obtain GAFF bonded and non-bonded parameters, (Case, 2005) besides, AmberTools was also used to obtain AM1-BCC charges. (Jakalian, 2000; Jakalian, 2002). Consistent with the OPLS-AA and GAFF parametrizations, a cutoff distance of 1 nm was used for the Lennard-Jones (LJ) and coulombic interactions. The AMOEBA force field parameterization of DIPA was done using the Poltype 2 package. (Walker, 2022).

To calculate the thermodynamic properties of pure solvents, simulation boxes with 400 solvent molecules were run for a total of 40 ns (30 ns of equilibration followed by a production stage of 10 ns). Explicitly, based on these simulations, the density, volumetric thermal expansion coefficient, solvent-solvent binding free energy, and heat of vaporization were calculated. The volumetric thermal expansion coefficient (ap) was calculated by

$${α}_{p} = \frac{1}{V} {( \frac{\partial V}{\partial T} )}_{p} , \qquad ( 1 )$$

where T is the temperature, V is the volume of the system is, and the differential was evaluated numerically by simulating the solvent at 294, 298, and 303 K. The heat of vaporization (ΔH^{vap}) was calculated based on the pure liquid solvent internal potential energy (Uₗ) using

$$Δ {H}^{v a p} = {U}_{g} - {U}_{l} + R T , \qquad ( 2 )$$

where R is the ideal gas constant and U_{g} is the internal potential energy of the gas phase, obtained by performing a simulation of an isolated molecule. To estimate the solvent-solvent binding free energy (w) the study used

$$w ( r ) = RT ln ( g ( r ) ) \qquad ( 3 )$$

where g(r) is the solvent-solvent radial distribution function (RDF).

The molecular interactions between solvent and water were explored through molecular dynamics simulations of mixtures comprising 400 solvent molecules and 400 water molecules, maintaining a 1:1 molar ratio. Similarly to the pure solvent systems, the bulk water-solvent mixtures were simulated for a total of 40 ns (30 ns of equilibration followed by 10 ns of production). Herein, gmx hbonds tools was used to calculate the average number of hydrogen-bonded (HB); in molecular dynamics framework, an HB is defined as hydrogen-donor-acceptor pair within a cutoff radius of 0.35 nm at an angle of 35 degrees. The solvent-water binding energy was also estimated using Eq. 3.

The solvation free energy of water in the amine-based solvents

$$( {μ}_{w}^{∞} )$$

was calculated by incrementally coupling the water molecule in the final configuration of the bulk pure solvent simulations; similarly, the same approach was used to calculate the solvent in water solvation free energy

$$( {μ}_{s o l v}^{∞} ) .$$

To do this, the coupling parameters for van der Waals and coulombic interactions were λ^{vdw/coul}={0.0, 0.2, 0.4, 0.6, 0.8, 1.0}, and the multistate Bennett acceptance ratio estimator (MBAR), available in Alchemical Analysis package, (Klimovich, 2015), was used to estimate the solvation free energy. (Shirts, 2008; Shirts, 2003; Bennett, 1976).

Following the recommendations of Klimovich et al., 1 ns of equilibration followed by 5 ns of production was enough to obtain accurate estimates.

The properties described above were used in the clustering step, based on the k-means clustering method, (Kubat, 2021; Rebala, 2019), in which all features were standardized using a z-score

$${z}_{i j} = \frac{{x}_{i j} - {\overline{x}}_{j}}{{σ}_{{x}_{j}}} , \qquad ( 4 )$$

where \tilde{x}ⱼ and σₓ_{ⱼ} are the average and standard deviation of the jth thermodynamic property, and xᵢⱼ is the jth thermodynamic property of the ith solvent.

Finally, in the interfacial simulations, the systems were equilibrated for 300 ns, followed by 100 ns for production purposes. The water-solvent interfacial systems were used to calculate the average density profiles across the simulation box from which the composition of the solvent-rich and aqueous phases were directly calculated. (Barbosa, 2022)

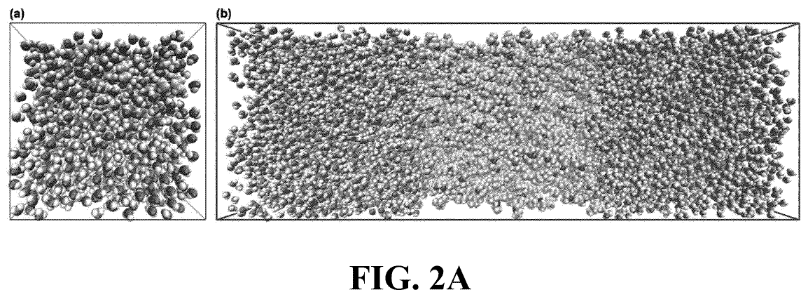

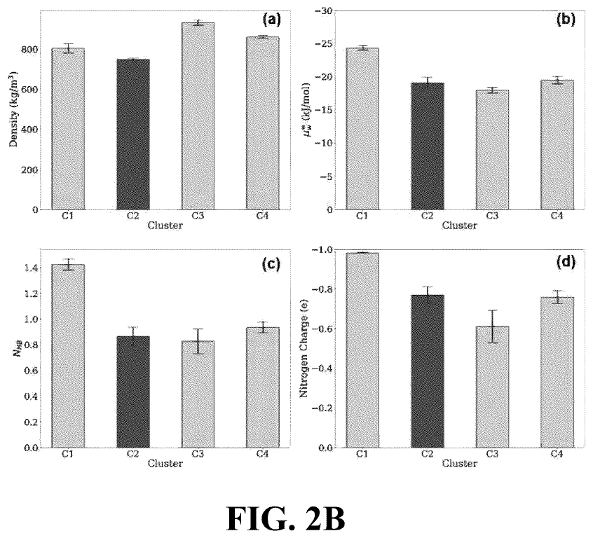

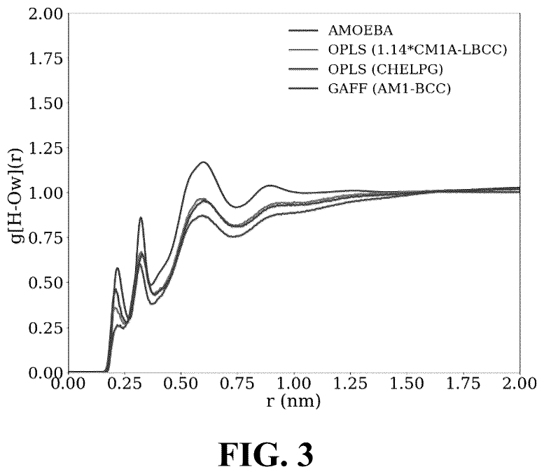

It is worth highlighting that classical molecular dynamics (MD) is a purely predictive modeling approach, which does not typically require experimental data fitting, and it only requires moderate computational resources. Nonetheless, it is advantageous to rigorously benchmark the accuracy of these molecular models against established experimental results or more sophisticated modeling methods for validation. In this approach, the study compared classical force fields, often referred to as fixed point charge models, and polarizable force fields, such as the widely recognized AMOEBA (Atomic Multipole Optimized Energetics for Biomolecular Applications).¹⁵ It is worth highlighting that despite the superior capability of polarizable force fields (e.g., AMOEBA) in accurately predicting thermodynamic and structural properties,¹⁶⁻¹⁷ their significant computational demands present a practical limitation. This is particularly pertinent when screening many solvents, as the heightened computational costs of polarizable force fields often outweigh their enhanced precision, guiding us toward a more balanced approach in selecting suitable modeling techniques for extensive solvent analysis. FIG. 3 shows the radial distribution function (g(r)) between the hydrogen site bonded to nitrogen (H) and the oxygen site of water (Ow), obtained by simulating an MD box with 2,000 water molecules and 30 M0 molecules (FIG. 2A, panel a). The OPLS-AA force field¹⁸⁻²⁰ using 1.14*CM1A-LBCC atomic partial charges¹⁹ and General Amber Force Field (GAFF)²¹ using the atomic partial charges obtained using the AM1-BCC method^{22,23} presented a better agreement with the g(r) predicted by the AMOEBA force field, which suggests a good performance of these force fields to describe the structure of DIPA in water.

The study further investigated the performance of OPLS-AA and GAFF (two of the most common intermolecular force fields) by benchmarking the predicted liquid-liquid phase equilibrium for M0 and dipropylamine (M1) against the data obtained experimentally by Davison.²⁴ To do this, interfacial system simulation boxes were built (FIG. 2A, panel b), in which the composition of each phase, solvent-rich and aqueous phases, are directly obtained. Table 1 shows the obtained results by using OPLS-AA (1.14*CM1A-LBCC). As observed, this force field prediction is in close agreement with the experimental data in this temperature range. This suggests that this modeling approach can reasonably predict the liquid-liquid phase equilibrium for amine-based solvents. Although GAFF performs well when predicting structural properties, as discussed before, the interfacial simulation suggested no water solubility. For this reason, OPLS-AA (1.14*CM1A-LBCC) was determined to be the ideal force field for these systems, and it was used to gather all of the simulation “production” data reported here.

TABLE 1
Predicted and experimental liquid-liquid equilibria for
M0 and M1 using OPLS-AA (1.14*CM1A-LBCC). Experimental
values obtained by Davison are presented in parentheses.

| | |
|---|---|
| Temp. | Solvent Molar fraction |

| Solvent | (K) | Water-rich phase | Solvent-rich phase |
|---|---|---|---|
| DIPA | 303 | 0.009 (0.021) | 0.276 (0.215) |
| | 313 | 0.005 (0.013) | 0.435 (0.408) |
| DPA | 303 | 0.006 (0.007) | 0.495 (0.425) |
| | 313 | 0.005 (0.005) | 0.574 (0.498) |

As highlighted above, M0 is often considered one of the best current solvents for TSSE desalination. To provide a more rigorous evaluation of these features, the k-means clustering method was used. This unsupervised learning (clustering) method aims to partition N observations into K clusters, mainly based on an optimization step of the so-called inertia criteria.^{25,26} The approach for screening a large number of amine-based solvents in the clustering stage prioritizes the use of thermodynamic properties which require low computational cost to obtain. Based on the results obtained by using the k-means method, the solvents affiliated with the M0 cluster were further evaluated for TSSE desalination performance by simulating their properties in an interfacial model.

As mentioned, the experiment started by predicting pure solvent properties, such as density, volumetric thermal expansion coefficient, solvent-solvent binding free energy, the heat of vaporization, number of H-bonded (N_{HB}) sites, and the solvation free energy of water in the amine solvents

$$( {μ}_{w}^{∞} ) .$$

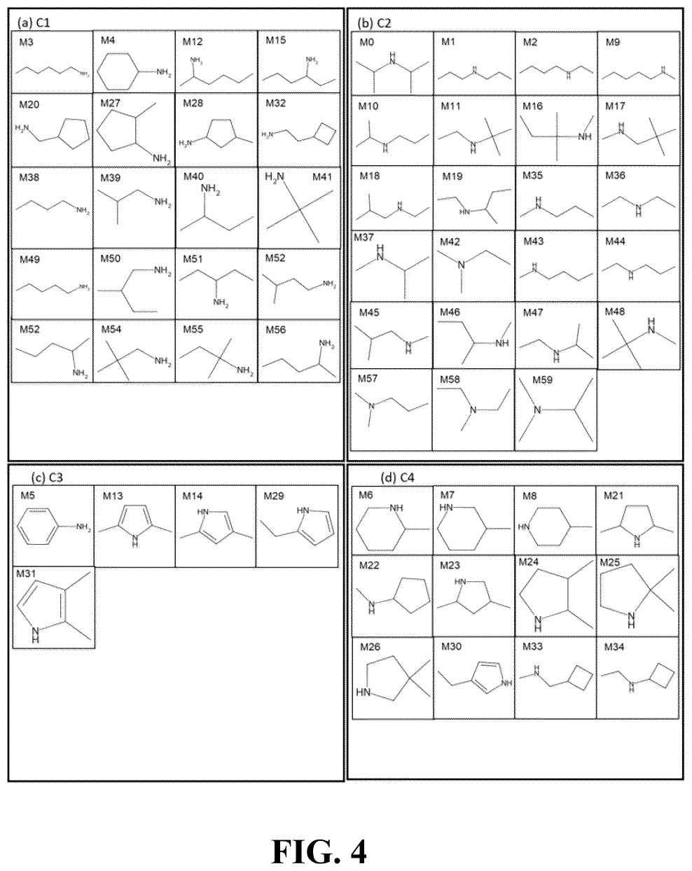

The pure fluid thermodynamic properties and thermodynamic and structural properties of water-solvent mixtures are summarized in Table 8 and Table 9, respectively; the clustering analysis results based on the thermodynamic properties of Table 8 and 9 are presented in FIG. 4.

The clustering analysis yields four clusters with distinct molecular topologies: (a) cluster C1 (M50 geometric center) is made up of all primary (1°) amines; (b) cluster C2 (M19 geometric center) is composed of 2° and tertiary (30) amines with shorter alkyl chains, including M0; (c) cluster C3 (M13 geometric center) is purely comprised of cyclic unsaturated amines; and (d) cluster C4 (M8 geometric center) mainly consists of unsaturated cyclic solvents. FIG. 2B shows the average solvent properties of each cluster: density,

$${μ}_{w}^{∞} ,$$

N_{HB}, and the nitrogen site partial charge. As shown, the densities of C1 and C4 are similar, while C2 and C3 correspond to the lowest and the highest average densities, respectively. Concerning the water solvation behavior in C1, C2, C3, and C4, FIG. 2B, panel b indicates that water solvation is most favorable in the C1 cluster (1° amines). At the same time,

$${μ}_{w}^{∞}$$

predicts similar solvation behavior in C2, C3, and C4, in which there is a slightly more favorable solvation of water in C4 solvents. Interestingly, the average number of HBs agrees with the overall behavior predicted by

$${μ}_{w}^{∞} ;$$

a more significant number of HB sites is found in C1 solvents, followed by C4, while similar behavior is observed for C2 and C3. Although not included as input in the k-means method clustering, the average number of H-bonds and the partial charge on the nitrogen sites are slightly correlated. For instance, the solvents with more electronegative nitrogen sites are assigned to C1, while those with less electronegative sites are found in the C3 cluster, and a smaller difference is observed between C2 and C4.

Now, based on the clustering analysis performed above, the study proceeded to analyze the amine solvent water uptake. In this analysis, solvent molecules were targeted that are grouped within the M0 cluster (C2) since M0 has already been identified as a promising candidate for the TSSE process, as mentioned before. Consistent with the MD prediction benchmark presented in Table 1, the interfacial simulations were carried out at 303 and 313 K; additional simulations were performed at 343 K for promising solvents, consistent with the typical water recovery temperature range for the TSSE process.

Table 2 presents the predicted liquid-liquid equilibrium for the amine-based solvents of C2. In this approach, a global mass fraction of zₛₒₗᵥ=0.3 was used in order to optimize the computational cost of the interfacial systems' MD simulation. Considering the short-chain 2° amine of C2 (FIG. 4), the MD simulations predicted fully miscible behavior in the evaluated temperature range and global composition, which suggests that these solvents are not good candidates for TSSE application, and these results are not shown in Table 2. In the evaluated set, M11 and M19 presented the largest water uptake, followed by M0, M10, M9, M2, M1, and M18. Beyond water uptake at room temperature, another relevant solvent feature for TSSE is composition temperature sensitivity. As observed, M0 presented a more pronounced ΔX₃₁₃/X₃₀₃ and ΔX₃₄₃/X₃₀₃, in which ΔXᵢ=Xᵢ−X₃₀₃ and Xᵢ is the solvent mole fraction at 303, 313, or 343 K. Notably, the high solubility sensitivity with temperature is in strong agreement with the good performance of M0 in the TSSE process previously reported on literature, as highlighted above. However, Table 2 also suggests a good performance of some of the six-carbon amine-based solvents, such as M11, M19, M10, M9, and M1, as it is possible to observe by looking at ΔX₃₁₃/X₃₀₃ and ΔX₃₄₃/X₃03; meanwhile, M2 and M18 were predicted not to perform well.

Finally, it was stated before that shorter-chain 2° amine solvents of C2, such as M44 and M46, are predicted to be completely miscible in water. Without wishing to be bound by theory, it was hypothesized that mixing these solvents could partially improve the performance of a six-carbon amine-based solvents such as M0. To explore this possibility, interfacial system simulations were run using the same global composition (zₛₒₗᵥ=0.3, z_{w}=0.7) and a mixture molar solvent fractions of x_{solv,M0}=0.8 and x_{solv,M44/M46}=0.2. Table 2 shows the predicted phase behavior for the mixture M0+M44 and M0+M46. By mixing these shorter-chain solvents with M0, a slightly higher water uptake is predicted in the solvent-rich phase for both mixtures. Considering the temperature sensitivity of solubility, a better performance is predicted for the mixture M0+M46 compared with M0 and the mixture M0+M44. These MD predictions suggest that the mixture of M0 with a shorter-chain amine-based could lead to a better TSSE performance.

TABLE 2
Predicted liquid-liquid equilibrium of different amine
solvents. Herein, ΔXᵢ = Xᵢ − X₃₀₃, in which
Xᵢ is the solvent molar fraction at 303, 313, or 343
K. All calculations were performed at 1 bar.

| | | Solvent Molar fraction | | | |
|---|---|---|---|---|---|
| | Temp. | Water-rich | Solvent-rich | ΔX₃₁₃/X₃₀₃ | ΔX₃₄₃/X₃₀₃ |
| Solvent | (K) | phase | phase | (%) | (%) |
| M0 | 303 | 0.009 | 0.276 | 50.104 | 176.051 |
| | 313 | 0.005 | 0.414 | | |
| | 343 | 0.003 | 0.761 | | |
| M1 | 303 | 0.007 | 0.495 | 15.972 | 44.416 |
| | 313 | 0.005 | 0.574 | | |
| | 343 | 0.002 | 0.715 | | |
| M2 | 303 | 0.004 | 0.440 | 8.237 | — |
| | 313 | 0.005 | 0.476 | | |
| M9 | 303 | 0.001 | 0.381 | 18.242 | 80.484 |
| | 313 | 0.002 | 0.451 | | |
| | 343 | 0.001 | 0.688 | | |
| M10 | 303 | 0.008 | 0.364 | 15.674 | 85.472 |
| | 313 | 0.006 | 0.421 | | |
| | 343 | 0.003 | 0.676 | | |
| M11 | 303 | 0.012 | 0.236 | 25.178 | 150.512 |
| | 313 | 0.008 | 0.295 | | |
| | 343 | 0.005 | 0.591 | | |
| M18 | 303 | 0.006 | 0.506 | 12.888 | — |
| | 313 | 0.004 | 0.571 | | |
| M19 | 303 | 0.008 | 0.235 | 31.201 | 130.571 |
| | 313 | 0.007 | 0.308 | | |
| | 343 | 0.004 | 0.541 | | |
| M44 + | 303 | 0.0103 | 0.2245 | 29.570 | 168.720 |
| M0 | 313 | 0.0076 | 0.2908 | | |
| | 343 | 0.0037 | 0.6032 | | |
| M46 + | 303 | 0.009 | 0.238 | 41.393 | 188.808 |
| M0 | 313 | 0.007 | 0.337 | | |
| | 343 | 0.004 | 0.689 | | |

Experimental screening of C6-solvents. The following solvents were tested experimentally to understand the effect of the nitrogen location on 6-carbon amine solvents: DIPA, dipropylamine (DPA), ethylbutylamine (EPA), methylpentylamine (MPA), hexylamine (HA), cyclohexylamine (CHA), and aniline. All of these solvents have a reported pKa (pulled from PubChem) of 10.6-11.07, except for aniline, which has a pKa of 4.6. Each solvent was mixed with DI water or brine at room temperature, allowed to extract for 1 hour and phase separated. Then, the solvent phase was put in a water bath at 70° C. for 1 h and the resulting phases were separated. Aniline showed no measurable performance in any of the tests performed, and therefore is not used in any of the experiments described below.

First, the study tested the solvents with DI water (i.e., no salt) to investigate the water uptake and release properties of each solvent. Additionally, the study tested for the temperature change upon mixing. Table 3 shows the results of the DI water mixing results. The water mass recovered was calculated using equation 5 where m_{wr} is the mass of water recovered, m_{wi} is the initial mass of water added to the solvent, and m_{w70} is the mass of water recovered after releasing from the solvent at 70° C.

$${m}_{w r} = \frac{{m}_{w 70}}{{m}_{w i}} \times 100 \qquad ( 5 )$$

TABLE 3
Water mass recovered (%) and temperature change of mixing
(° C.) of the solvents with DI water using a 1:1
volume ratio of solvent:water. n ≥ 3 unless otherwise noted.

| | Water Mass | Mixing |
|---|---|---|
| Solvent | Recovered (%) | ΔT (° C.) |
| HA | 36.6 ± 0.8 | 8.3 ± 0.1 |
| MPA | 40.7 (n = 1) | 8.8 (n = 1) |
| EBA | 21.5 (n = 1) | 7.6 (n = 1) |
| DPA | 14.0 ± 0.2 | 6.8 ± 0.7 |
| DIPA | 40.5 ± 1.8 | 8.1 ± 0.5 |
| CHA | 0.0 ± 0.0 | 10.1 ± 0.3 |
| Aniline | 0.0 ± 0.0 | −0.9 ± 0.5 |

Next, the study investigated the effect of change in volume upon mixing the solvent and either DI water or a 100,000 ppm NaCl brine. 4 mL of solvent was added to 4 mL of the aqueous phase at room temperature (20-22° C.). If these mixtures behave ideally, the volumes would be additive to 8 mL. Since the solvent/organic phase will take in water, the volume of the mixture will be reduced. Table 4 presents the volume of mixing results with DI water, and Table 5 presents the results with the NaCl brine. The DI water results do not show much difference as the solvents all perform similarly, except for HA and CHA, which are both miscible with the aqueous phase. The NaCl brine testing demonstrates that HA and CHA are not suitable solvents since HA forms a gel and CHA forms a one phase mixture. MPA had the most different results with a lower aqueous volume and larger organic volume than the other solvents. EPA, DPA, and DIPA were all relatively similar with some small differences.

TABLE 4
Volume of mixing results with 4 mL of
solvent and 4 mL of DI water. n ≥ 3.

| | Mixing | Aqueous | Organic |
|---|---|---|---|
| Solvent | Volume (mL) | Volume (mL) | Volume (mL) |
| HA | 7.69 ± 0.09 | N/A (one phase) | N/A (one phase) |
| MPA | 7.65 ± 0.06 | 1.36 ± 0.05 | 6.29 ± 0.03 |
| EBA | 7.42 ± 0.03 | 2.77 ± 0.06 | 4.65 ± 0.05 |
| DPA | 7.54 ± 0.16 | 3.35 ± 0.26 | 4.19 ± 0.16 |
| DIPA | 7.54 ± 0.08 | 1.78 ± 0.03 | 5.75 ± 0.05 |
| CHA | 7.10 ± 0.00 | N/A (one phase) | N/A (one phase) |

TABLE 5
Volume of mixing results using 4 mL of solvent
and 4 mL of a 100,000 ppm NaCl brine. n ≥ 3.

| | Mixing | Aqueous | Organic |
|---|---|---|---|
| Solvent | Volume (mL) | Volume (mL) | Volume (mL) |
| HA | ~8 (gel formed) | N/A (gel) | N/A (gel) |
| MPA | 7.74 ± 0.06 | 2.13 ± 0.07 | 5.61 ± 0.01 |
| EBA | 7.53 ± 0.06 | 3.35 ± 0.05 | 4.18 ± 0.03 |
| DPA | 7.44 ± 0.21 | 3.53 ± 0.09 | 3.92 ± 0.18 |
| DIPA | 7.68 ± 0.03 | 3.18 ± 0.02 | 4.51 ± 0.05 |
| CHA | 7.23 ± 0.06 | N/A (one phase) | N/A (one phase) |

The study investigated if these ΔV and ΔT data could qualitatively indicate performance of the solvents for desalinating brines by testing all seven solvents for water recovery and desalination properties using a 100,000 ppm (1.71 M) NaCl brine. Table 6 shows the results. The results indicate there is a balance between water mass recovered and the salt rejection. While HA and CHA formed one phase at room temperature, separate phases were formed at 70° C. Their water mass recovered was significantly higher than the other solvents, but their salt rejections were negative, indicating an increase in salt concentration in the final “purified” water. Therefore, HA and CHA, which contain 10 amines and not 2° amines are not useful for TSSE. Aniline showed no results as expected with it being an aromatic 1° amine and more hydrophobic than the other solvents with a very different pKₐ value. MPA showed a higher water recovery, but the salt rejection was very low. With EBA, the water recovery reduced but the salt rejection increased by an order of magnitude compared to MPA. When the amine is moved to the center of the molecule, the salt rejection further increased. DIPA, the gold standard in the literature, also was the best performing solvent in these preliminary studies. The symmetry combined with the hindered amine appear to affect performance to some degree. Salt rejection was calculated using equation 6, where R is the rejection, C_{w70} is the concentration of salt in the water phase after 70° C., and C_{wi} is the concentration of salt in the initial water phase added to the solvent. The water mass recovered was calculated using equation 5 described above.

$$R = ( 1 - \frac{{C}_{w 70}}{{C}_{w i}} ) \times 100 \qquad ( 6 )$$

TABLE 6
NaCl rejection (%), water mass recovered (%), and temperature
change of mixing (° C.) of the solvents with 100,000
ppm NaCl brine. n ≥ 3.

| | | Water Mass | Mixing |
|---|---|---|---|
| Solvent | Rejection (%) | Recovered (%) | ΔT (° C.) |
| HA | −28.2 ± 15.0 | 50.4 ± 1.8 | 9.3 ± 0.6 |
| MPA | 7.7 ± 0.4 | 18.6 ± 0.3 | 8.0 ± 0.1 |
| EBA | 73.4 ± 5.4 | 6.5 ± 0.6 | 6.3 ± 0.1 |
| DPA | 88.9 ± 1.7 | 5.2 ± 0.7 | 4.6 ± 0.4 |
| DIPA | 83.6 ± 3.0 | 9.2 ± 0.5 | 5.6 ± 1.2 |
| CHA | −15.4 ± 2.5 | 25.9 ± 6.4 | 9.0 ± 1.0 |
| Aniline | N/A | 0.0 ± 0.0 | 0.3 ± 0.5 |

Discussion. Based on molecular simulation data, the study benchmarked 59 different amine-based solvents against DIPA (currently one of the best-performing TSSE solvents) for potential use in TSSE brine desalination. To do this, the accuracy of MD based on a classical force field was explicitly evaluated by benchmarking against a polarizable force field and experimental liquid-liquid phase equilibria data. After establishing the reliability of the MD models, the study performed TSSE solvent screening by using a database of pure solvent thermophysical and thermodynamic properties, such as density, heat of vaporization, volumetric thermal expansion coefficient, and solvent-solvent binding free energy (all obtained by MD simulations). To aid the screening process, the k-means method was used to cluster the different solvents based on their evaluated thermodynamic properties. By doing so, 1° amines, long-chain/saturated cyclic solvents, unsaturated cyclic solvents, and 2° linear and branched amines were separated into different clusters.

It was believed that solvents grouped within the cluster containing DIPA would be effective candidates for TSSE. Additional interfacial water-solvent simulations were performed involving the solvents within the DIPA cluster. The interfacial system simulations indicate good performance of 2° amines with six carbons in a branched or linear structure, although worse performance was predicted for N-ethylbutan-1-amine and N-ethyl-2-methylpropan-1-amine within the evaluated amine solvents. Finally, although certain short-chain amine solvents were not predicted to perform well (due to the water and solvent mutual solubility), certain mixtures (e.g., of diisopropylamine and N-methylbutan-2-amine) were unexpectedly shown to perform better than pure DIPA.

Experimentally, it was shown that the symmetry of the six-carbon amine-containing molecule assisted with salt rejection. It was demonstrated that hindered solvents seem to have the best performing results so far.

TABLE 7
Label, SMILES string, name, and molecular structure of the screened
amine-based solvents.

| Label | Smile | Name | Structure |
|---|---|---|---|
| M0 | CC(C)NC(C)C | diisopropylamine | 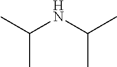 |
| M1 | CCCNCCC | dipropylamine | 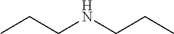 |
| M2 | CCCCNCC | N-ethylbutan-1-amine | 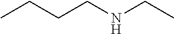 |
| M3 | CCCCCCN | hexan-1-amine | 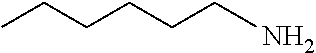 |
| M4 | NC1CCCCC1 | cyclohexanamine | 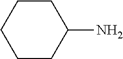 |
| M5 | NC1═CC═CC═C1 | aniline | 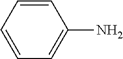 |
| M6 | CC1CCCCN1 | 2-methylpiperidine | 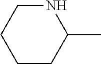 |
| M7 | CC1CCCNC1 | 3-methylpiperidine | 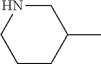 |
| M8 | CC1CCNCC1 | 4-methylpiperidine | 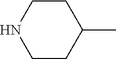 |
| M9 | CCCCCNC | N-methylpentan-1-amine | 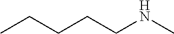 |
| M10 | CCCNC(C)C | N-isopropylpropan-1-amine | 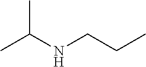 |
| M11 | CCNC(C)(C)C | N-ethyl-2-methylpropan-2-amine | 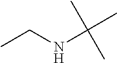 |
| M12 | CCCCC(N)C | hexan-2-amine | 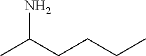 |
| M13 | CC1═CC═C(C)N1 | 2,5-dimethyl-1H-pyrrole | 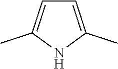 |
| M14 | CC1═CC(C)═CN1 | 2,4-dimethyl-1H-pyrrole | 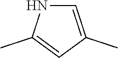 |
| M15 | CCCC(N)CC | hexan-3-amine | 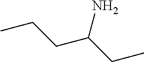 |
| M16 | CCCCCNC | N,2-dimethylbutan-2-amine | 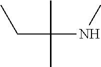 |
| M17 | CCCNC(C)C | N,2,2-trimethylpropan-1-amine | 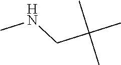 |
| M18 | CCNCC(C)C | N-ethyl-2-methylpropan-1-amine | 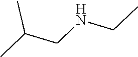 |
| M19 | CCNC(C)CC | N-ethylbutan-2-amine | 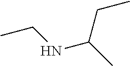 |
| M20 | NCC1CCCC1 | cyclopentylmenthanamine | 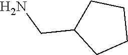 |
| M21 | CC1CCC(C)N1 | 2,5-dimethylpyrrolidine | 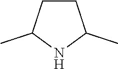 |
| M22 | CNC1CCCC1 | N-methylcyclopentanamine | 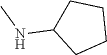 |
| M23 | CC1CC(C)CN1 | 2,4-dimethylpyrrolidine | 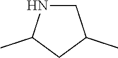 |
| M24 | CC1C(C)CCN1 | 2,3-dimethylpyrrolidine | 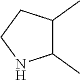 |
| M25 | CC1(C)CCCN1 | 2,2-dimethylpyrrolidine | 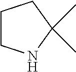 |
| M26 | CC1(C)CCNC1 | 3,3-dimethylpyrrolidine | 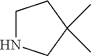 |
| M27 | NC1C(C)CCC1 | 2-methylcyclopentan-1-amine | 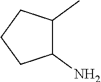 |
| M28 | NC1CC(C)CC1 | 3-methylcyclopentan-1-amine | 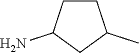 |
| M29 | CCC1═CC═CN1 | 2-ethyl-1H-pyrrole | 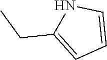 |
| M30 | CCC1═CNC═C1 | 3-ethyl-1H-pyrrole | 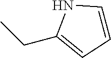 |
| M31 | CC1═C(C)C═CN1 | 2,3-dimethyl-1H-pyrrole | 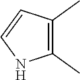 |
| M32 | NCCC1CCC1 | 2-cyclobutylethan-1-amine | 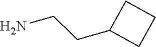 |
| M33 | CNCC1CCC1 | 1-cyclobutyl-N-methylmethanamine | 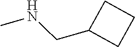 |
| M34 | CCNC1CCC1 | N-ethylcyclobutanamine | 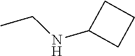 |
| M35 | CNCCC | N-methylpropan-1-amine | 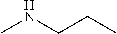 |
| M36 | CCNCC | diethylamine | 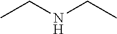 |
| M37 | CNC(C)C | N-methylpropan-2-amine | 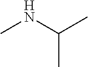 |
| M38 | NCCCC | butan-1-amine | 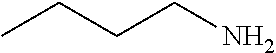 |
| M39 | CC(C)CN | 2-methylpropan-1-amine | 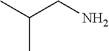 |
| M40 | NC(C)CC | butan-2-amine | 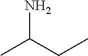 |
| M41 | CC(C)(N)C | 2-methylpropan-2-amine | 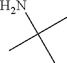 |
| M42 | CN(C)CC | N,N-dimethylethanamine | 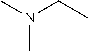 |
| M43 | CNCCCC | N-methylbutan-1-amine | 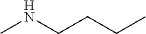 |
| M44 | CCNCCC | N-ethylpropan-1-amine | 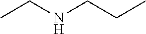 |
| M45 | CC(C)CNC | N,2-dimethylpropan-1-amine | 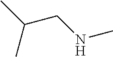 |
| M46 | CNC(C)CC | N-methylbutan-2-amine | 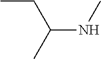 |
| M47 | CCNC(C)C | N-ethylpropan-2-amine | 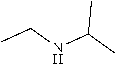 |
| M48 | CC(C)(C)NC | N,2-dimethylpropan-2-amine | 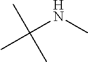 |
| M49 | CCCCCN | pentan-1-amine | 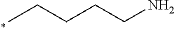 |
| M50 | NCC(C)CC | 2-methylbutan-1-amine | 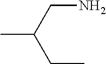 |
| M51 | CCC(N)CC | pentan-3-amine | 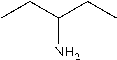 |
| M52 | CC(C)CCN | 3-methylbutan-1-amine | 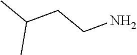 |
| M53 | CC(N)CCC | pentan-2-amine | 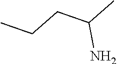 |
| M54 | CC(C)(C)CN | 2,2-dimethylpropan-1-amine | 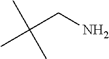 |
| M55 | CC(C)(N)CC | 2-methylbutan-2-amine | 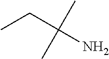 |
| M56 | C(C)CC(N)C | pentan-2-amine | 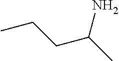 |
| M57 | CN(C)CCC | N,N-dimethylpropan-1-amine | 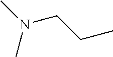 |
| M58 | CCN(C)CC | N-ethyl-N-methylethanamine | 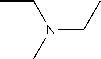 |
| M59 | CN(C)C(C)C | N-Ndimethylpropan-2-amine | 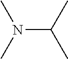 |

TABLE 8
Average density, volumetric thermal expansion coefficient
(αₚ), solvent-solvent binding free energy (w), fluid internal
potential energy (Uₗ), and heat of vaporization (ΔH^{vap})
evaluated at a temperature of 298 K.

| | Density | αₚ | W | Uₗ | ΔH^{vap} |
|---|---|---|---|---|---|
| Solvent | (kg/m³) | (10⁻²K⁻¹) | (kJ/mol) | kJ/mol | (kJ/mol) |
| M0 | 753.783 | 0.133 | −1.116 | −66.139 | 37.759 |
| M1 | 750.780 | 0.145 | −0.311 | 2.213 | 40.997 |
| M2 | 754.465 | 0.130 | −0.203 | 21.623 | 42.736 |
| M3 | 787.263 | 0.136 | −0.478 | 25.881 | 51.915 |
| M4 | 902.106 | 0.112 | −2.165 | −14.434 | 50.978 |
| M5 | 977.100 | 0.036 | −1.709 | −55.611 | 63.542 |
| M6 | 870.705 | 0.120 | −2.083 | 12.744 | 43.858 |
| M7 | 868.740 | 0.091 | −2.297 | 36.594 | 44.448 |
| M8 | 858.291 | 0.114 | −1.834 | 60.223 | 43.612 |
| M9 | 760.879 | 0.128 | −0.409 | 57.697 | 44.728 |
| M10 | 754.071 | 0.135 | −0.674 | −39.726 | 39.508 |
| M11 | 765.420 | 0.132 | −1.206 | −84.899 | 39.225 |
| M12 | 785.256 | 0.132 | −0.662 | −26.138 | 48.201 |
| M13 | 907.512 | 0.023 | −1.891 | −59.619 | 70.166 |
| M14 | 916.876 | 0.067 | −2.388 | −50.272 | 60.771 |
| M15 | 781.882 | 0.138 | −0.956 | −40.631 | 46.640 |
| M16 | 778.821 | 0.122 | −1.767 | −78.013 | 40.031 |
| M17 | 766.423 | 0.128 | −1.368 | 9.391 | 39.820 |
| M18 | 753.657 | 0.145 | −0.612 | −5.679 | 40.581 |
| M19 | 757.006 | 0.132 | −0.787 | −45.346 | 38.951 |
| M20 | 884.997 | 0.111 | −1.622 | 83.100 | 50.666 |
| M21 | 852.462 | 0.120 | −1.246 | 8.976 | 43.149 |
| M22 | 860.357 | 0.123 | −1.106 | 68.656 | 44.916 |
| M23 | 850.400 | 0.121 | −1.185 | 44.091 | 44.111 |
| M24 | 856.200 | 0.122 | −1.462 | 29.297 | 44.715 |
| M25 | 857.586 | 0.113 | −1.849 | −7.138 | 42.296 |
| M26 | 858.170 | 0.123 | −2.237 | 80.231 | 40.790 |
| M27 | 887.888 | 0.113 | −1.797 | −8.039 | 49.269 |
| M28 | 886.271 | 0.118 | −1.971 | 34.561 | 50.445 |
| M29 | 905.156 | 0.032 | −1.531 | −38.204 | 47.366 |
| M30 | 912.118 | 0.027 | −1.911 | −15.724 | 25.145 |
| M31 | 960.400 | 0.057 | −2.223 | −72.449 | 83.077 |
| M32 | 887.360 | 0.109 | −1.054 | 41.704 | 54.940 |
| M33 | 849.737 | 0.084 | −0.773 | 56.961 | 46.386 |
| M34 | 848.756 | 0.105 | −0.708 | 6.100 | 45.554 |
| M35 | 731.120 | 0.161 | −0.635 | 34.089 | 33.226 |
| M36 | 729.932 | 0.168 | −0.614 | 1.409 | 32.151 |
| M37 | 738.080 | 0.156 | −1.214 | −13.503 | 32.679 |
| M38 | 764.234 | 0.153 | −1.066 | 14.830 | 39.880 |
| M39 | 767.428 | 0.147 | −1.793 | −29.764 | 35.842 |
| M40 | 765.104 | 0.148 | −1.390 | −54.446 | 36.543 |
| M41 | 766.052 | 0.156 | −1.815 | −97.893 | 34.996 |
| M42 | 722.271 | 0.148 | −1.520 | 35.732 | 30.975 |
| M43 | 747.083 | 0.142 | −0.480 | 53.462 | 39.160 |
| M44 | 741.830 | 0.141 | −0.453 | 1.776 | 37.522 |
| M45 | 747.509 | 0.148 | −0.908 | 25.406 | 36.974 |
| M46 | 752.835 | 0.137 | −1.133 | −17.959 | 36.149 |
| M47 | 745.181 | 0.153 | −0.829 | −39.136 | 37.218 |
| M48 | 764.143 | 0.147 | −1.839 | −65.703 | 35.399 |
| M49 | 776.892 | 0.137 | −0.643 | 18.855 | 46.416 |
| M50 | 780.899 | 0.143 | −1.546 | −10.144 | 42.900 |
| M51 | 773.232 | 0.145 | −1.129 | −65.944 | 39.793 |
| M52 | 784.341 | 0.133 | −1.054 | 31.718 | 45.498 |
| M53 | 775.514 | 0.146 | −1.062 | −25.327 | 42.793 |
| M54 | 787.933 | 0.140 | −2.501 | −58.253 | 39.784 |
| M55 | 782.712 | 0.139 | −1.631 | −119.562 | 38.915 |
| M56 | 774.562 | 0.135 | −1.038 | −28.627 | 41.399 |
| M57 | 737.991 | 0.126 | −1.053 | 47.235 | 34.738 |
| M58 | 741.798 | 0.133 | −1.330 | 7.673 | 35.736 |
| M59 | 747.955 | 0.128 | −1.842 | −8.025 | 34.103 |

TABLE 9
$Solvation free energy of water in the evaluated solvents ( {μ}_{w}^{∞} ) , solvent - water$
binding free energy (w^{sol-w}), the average number of solvent-water H-bonds per solvent
molecule (N_{HB}), and the average number of sites solvent-water within the H-bond cutoff
radius not H-bonded per solvent molecule (N_{NHB}) for the mixture of the solvents with water
at 298K.

| | ${μ}_{w}^{∞}$ | w^{sol-w} | | |
|---|---|---|---|---|
| Solvent | (kJ/mol) | (kJ/mol) | N_{HB} | N_{NHB} |
| M0 | −17.88 | −2.93 | 0.85 | 0.96 |
| M1 | −19.16 | −2.68 | 0.90 | 1.02 |
| M2 | −19.43 | −2.08 | 0.98 | 1.13 |
| M3 | −24.61 | −0.54 | 1.54 | 2.35 |
| M4 | −23.41 | −1.66 | 1.50 | 2.27 |
| M5 | −19.51 | −1.13 | 1.23 | 1.77 |
| M6 | −17.35 | −1.08 | 0.88 | 1.13 |
| M7 | −19.90 | −1.58 | 0.92 | 1.16 |
| M8 | −18.45 | −1.18 | 0.97 | 1.28 |
| M9 | −19.26 | −1.07 | 0.97 | 1.17 |
| M10 | −19.66 | −2.74 | 0.90 | 1.01 |
| M11 | −18.47 | −2.84 | 0.88 | 0.99 |
| M12 | −22.99 | −0.99 | 1.38 | 2.07 |
| M13 | −18.09 | −1.68 | 0.79 | 1.07 |
| M14 | −18.11 | −1.39 | 0.68 | 1.01 |
| M15 | −22.34 | −1.42 | 1.29 | 1.99 |
| M16 | −19.22 | −1.74 | 0.92 | 1.09 |
| M17 | −18.20 | −1.73 | 0.81 | 0.95 |
| M18 | −18.78 | −2.45 | 0.83 | 0.93 |
| M19 | −19.40 | −2.14 | 0.89 | 1.00 |
| M20 | −24.44 | −1.12 | 1.50 | 2.27 |
| M21 | −20.42 | −1.82 | 0.98 | 1.17 |
| M22 | −19.84 | −1.16 | 1.01 | 1.21 |
| M23 | −20.58 | −0.81 | 1.01 | 1.25 |
| M24 | −20.90 | −0.81 | 1.03 | 1.26 |
| M25 | −20.24 | −1.76 | 0.95 | 1.16 |
| M26 | −18.61 | −1.58 | 0.89 | 1.15 |
| M27 | −25.37 | −1.64 | 1.46 | 2.20 |
| M28 | −24.67 | −1.72 | 1.50 | 2.26 |
| M29 | −17.64 | −0.76 | 0.76 | 1.09 |
| M30 | −17.07 | −0.97 | 0.67 | 1.11 |
| M31 | −16.48 | −1.30 | 0.68 | 1.01 |
| M32 | −24.22 | −0.98 | 1.57 | 2.39 |
| M34 | −20.26 | −1.81 | 0.95 | 1.09 |
| M35 | −20.96 | −1.54 | 1.00 | 1.19 |
| M36 | −21.97 | −2.56 | 1.02 | 1.17 |
| M37 | −21.80 | −1.66 | 1.02 | 1.21 |
| M38 | −25.12 | −1.00 | 1.52 | 2.30 |
| M39 | −25.65 | −1.63 | 1.38 | 2.08 |
| M40 | −25.27 | −1.73 | 1.38 | 2.07 |
| M41 | −25.09 | −2.06 | 1.37 | 2.00 |
| M42 | −16.84 | −1.72 | 0.60 | 0.02 |
| M43 | −20.03 | −1.15 | 0.99 | 1.20 |
| M44 | −20.23 | −2.52 | 0.97 | 1.10 |
| M45 | −20.10 | −1.46 | 0.90 | 1.07 |
| M46 | −20.61 | −1.28 | 0.94 | 1.12 |
| M47 | −20.48 | −2.49 | 0.96 | 1.08 |
| M48 | −20.12 | −2.17 | 0.97 | 1.15 |
| M49 | −24.36 | −0.86 | 1.53 | 2.31 |
| M50 | −24.11 | −1.52 | 1.40 | 2.13 |
| M51 | −24.00 | −1.61 | 1.28 | 1.96 |
| M52 | −24.70 | −1.12 | 1.54 | 2.35 |
| M53 | −24.15 | −1.42 | 1.42 | 2.15 |
| M54 | −24.11 | −2.02 | 1.27 | 1.98 |
| M55 | −24.02 | −2.17 | 1.28 | 1.90 |
| M56 | −24.44 | −1.34 | 1.39 | 2.09 |
| M57 | −15.22 | −0.91 | 0.53 | 0.01 |
| M58 | −15.11 | −1.54 | 0.54 | 0.01 |

### Example 2: Glycerol-Derived Solvents

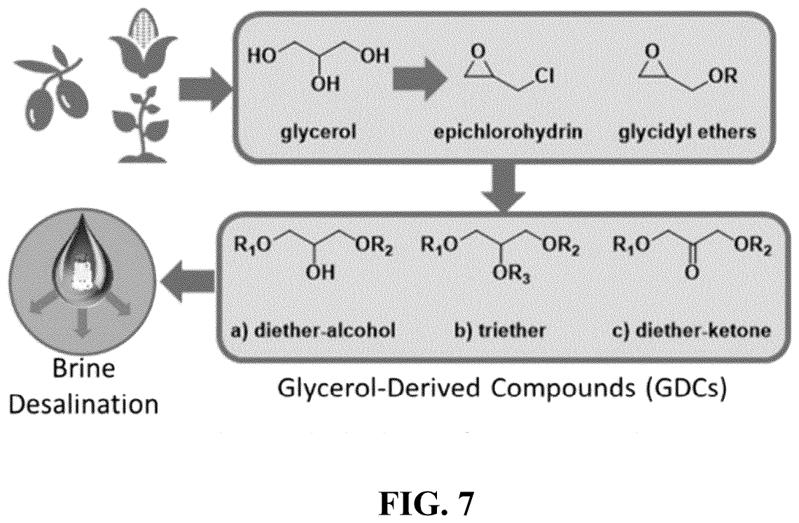

Synthesizing Glycerol Derived Compounds. In many processes/applications, GDCs can be replacements for conventional organic solvents, while simultaneously offering enhancements arising from their low viscosity, tunable hydrophilicity/hydrophobicity, solvating power, and green origins.^{11-12, 44-45} The general structure of several types of GDCs are displayed in FIG. 7 along with functional groups (R¹, R², and R³) that can be independently selected to control the physical, chemical, and thermodynamic properties of GDCs. The study has observed that although water can dissolve in GDCs, most GDCs have limited solubility in water, which is a highly advantageous behavior for designing effective solvents for TSSE. Epichlorohydrin is a convenient starting material, as it is a primary commodity chemical derived from glycerol but more reactive.²²⁻²³ Glycidyl ethers also provide useful starting materials for the synthesis of GDCs for TSSE, and certain glycidyl ethers are already commercially available in large quantities. Furthermore, the production of GDCs directly from glycerol simultaneously eliminates the requirement for use of chlorinated reagents. Compounds with symmetric (e.g., R¹=R²=R³) and asymmetric functional groups are synthesized and characterized to develop a rigorous understanding of the impact of functional group type and placement on relevant properties. From this, the impact of ‘R’ groups on TSSE performance in GDC diether-alcohols (FIG. 7, panel a), triethers (FIG. 7, panel b), and diether-ketones (FIG. 7, panel c) can be measured. Furthermore, glycidyl ethers and amines are utilized to form GDCs with 2° amine groups to study solvents which compare directly to amines like DIPA. An example of this chemistry is shown in Scheme 1.

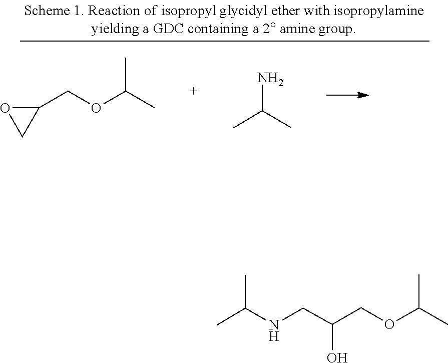

Functional groups are modified to observe the effect the various groups impact specific performance targets, including combinations of properties such as high solubility of water, hydrophobicity/hydrophilicity, low ion solvation, and low viscosity.⁵⁰

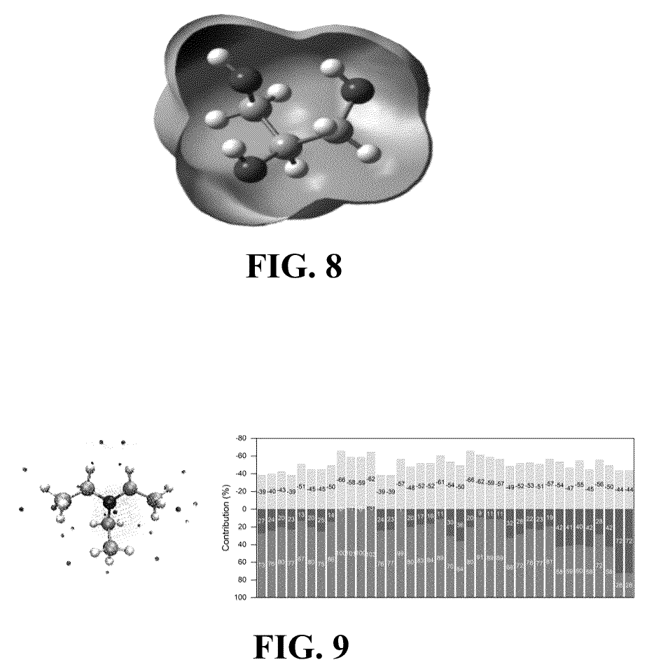

Density Functional Theory Screening of GDCs. A suite of computational tools and descriptors have been developed that can be used to accelerate the investigation of large libraries of compounds and their thermophysical properties. Several different correlations have been developed that can be used to extract information from the electrostatic surface properties of individual molecules, based on first-principles density functional theory (DFT).⁵⁴ Since the GDCs studied in this example are relatively small molecules comprised of light elements, the DFT calculations are fast (glycerol shown in FIG. 8 required a total of 9 CPU minutes for complete structural convergence). The resulting electrostatic properties are analyzed in terms of polarity (dipole moment, polarizability, etc.), and statistical analyses of the electrostatic potential were performed to make more detailed predictions about estimated solvation properties, viscosities, etc. These calculations can be easily automated to explore permutations in (R₁, R₂, and R₃)-groups by generating a library of compounds as SMILES strings and feeding them into the DFT calculation engine.

Efficient and rigorous protocols for searching and optimizing small molecule structures by using a hierarchy of screening/relaxation stages have been developed. For instance, a large set of possible molecular structures are initially generated using the Molclus program.⁵⁵ These structures then serve as initial configurations for semi-empirical quantum mechanical optimizations using CREST⁵⁶ and GFNO-xTB⁵⁷⁻⁵⁸ within the xTB program.⁵⁷ The relaxed structures are further optimized at a higher level with GFN2-xTB.⁵⁹ Next, single-point energy calculations of the lowest-energy configurations are performed at the B97-3c level⁶⁰ using ORCA 4.2.0⁶¹⁻⁶² to extract fundamental properties/interactions (see FIG. 9).

Beyond just prediction/screening of the GDC thermophysical properties, it is also possible to provide more detailed information about the solvation mechanisms in these systems. These mechanistic details provide direct feedback about the most active sites within the GDC molecules, and thus, provide more rational synthetic pathways for tuning the solvent behavior.

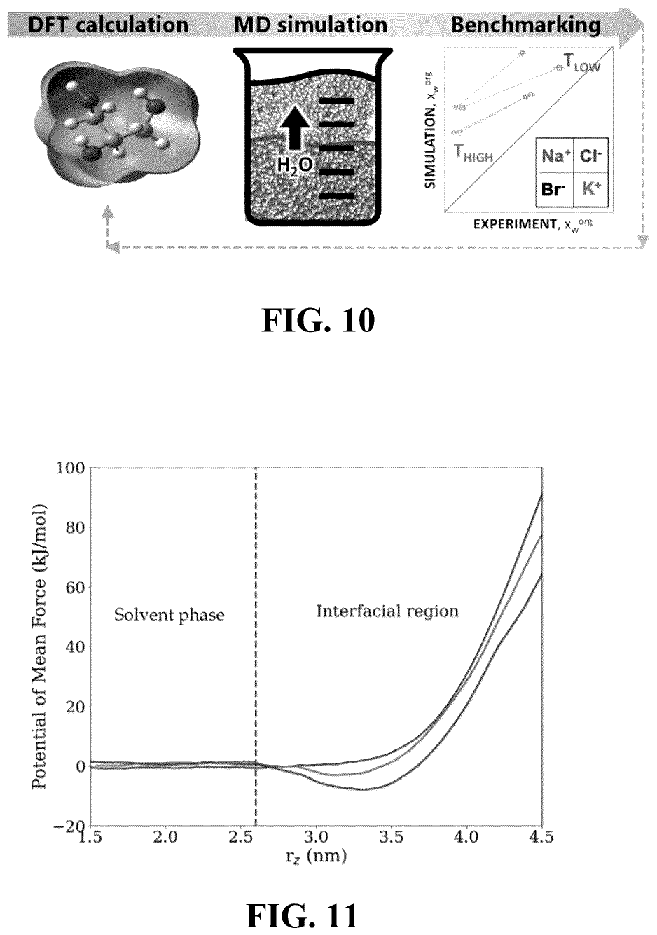

Molecular Dynamics Screening of GDCs. Following the DFT-level screening of the individual molecules, larger-scale tests are performed via molecular dynamics (MD) simulations. Instead of merely analyzing a single isolated solvent molecule, the MD simulations allow the capture of a realistic bulk liquid environment. From short simulations of the bulk solvents, accurate estimates of the thermophysical properties of the system are made. Similar to the electrostatic analyses of the individual molecules, MD analyses are performed on the larger systems (i.e., 100s-1000s of molecules). Once equilibrated, instantaneous snapshots of the void spaces are taken in the solvent to generate a statistical representation of the void structure, and superimpose the electrostatic potential onto these void spaces in order to quantify the local electrostatic environment (felt by a solute molecule).⁶³⁻⁶⁵ These screening procedures are more efficient than directly calculating chemical potentials of different solutes or performing grand canonical Monte Carlo simulations. This screening hierarchy is used to make reliable predictions of properties such as Hansen solubility parameters, which can then be directly connected to experimental measurements and used to reduce experimental effort needed to identify the most promising molecules. FIG. 10 shows there will be a continuous feedback loop between computation and experiment to converge to optimized molecules (performance and economics) for brine desalination.

As the final stage in the simulation and modeling protocol, two-phase (i.e., organic+aqueous phases) MD simulations are performed of the liquid-liquid equilibria behavior. Since these systems involve multiple components and multiple phase equilibria, they are the most computationally demanding, but they provide the most representative model of the experimental system. One advantage of this model is that it promotes the direct tracking of the water transport from the brine phase to the solvent phase as a function of temperature, as well as solvent contamination in the aqueous phase. Furthermore, salt crystallization in the brine phase can be monitored, as the water molecules are depleted (raising the salt concentration).

The two-phase MD simulations have been tested with several different salt species (NaCl, KBr, KCl, and NaBr). Experimental benchmarking has confirmed that the MD simulations provide excellent qualitative performance predictions, with elevated values of water absorption in the solvent phase.⁴² Furthermore, the simulations indicate that a dominant water absorption mechanism within the solvent phase is via hydrogen-bonding interactions—thereby identifying significant metrics for screening prototype solvent candidates.

In the two-phase MD simulations, the evaluation is extended beyond just the equilibrium thermodynamics and structures of these systems. The potential-of-mean force (PMF) required to transfer a water molecule from the brine phase to the solvent phase can also be calculated. This type of analysis provides a quantitative value for the free energy barrier (i.e., an estimate of the rate) to transfer a water molecule between phases (see FIG. 11). Similar to previous work,^{41,42} the distance between the center-of-mass of the water molecule and the solvent phase will be used as the reaction coordinate. The umbrella method and the weighted histogram analysis method are used to estimate the free energy based on the biased PMF simulations. A spacing of 0.15 nm between each simulation window and a spring constant of 1000 kJ/(mol·m²) is sufficient to obtain an acceptable overlap of probability distributions.

The DFT-level screening is used to down-select solvent candidates, based on electrostatic descriptors, and the bulk solvent MD simulations will be used to further down-select the solvent candidates (based on bulk solvent structures, heat capacity, solvation free energies, etc.). These calculations are relatively fast, and much of the analysis can be automated. While it can be challenging to obtain quantitative predictions from these calculations, it has been shown²⁷ that they are very effective for qualitative screening purposes (saving significant experimental time and expense). The two-phase MD simulations can as the primary bridge to the experimental systems, since tests analogous to the experimental performance evaluations can be performed. The models are designed to mimic the experimental testing protocols (brine-to-solvent phase volume ratios, brine compositions, temperature shifts, etc.). Output from these two-phase MD simulations provide critical information about the viability of the solvents for experimental testing. This includes water composition in solvent (at low and high temperatures), fraction of solvent in brine phase, composition of different species at the solvent-brine interface, etc.

Performance evaluate GDCs for brine desalination in TSSE using a NaCl brine. A protocol has been developed for solvent performance testing for brine desalination.³⁴ Solvent performance testing is done using temperature-controlled water baths. Solvents are used at room temperature (˜20-25° C.). The solvent is mixed with an equal volume of a 100 g/L (1.7 mol/L) NaCl solution. The solutions are allowed to equilibrate for 0.5-1 h. The solvent are separated from the concentrated brine using pipets. The water-laden solvent are heated to three temperatures (50, 65, 80° C.) to cause phase separation. The solvent and water phases are then separated using pipets. Promising solvents are tested for recyclability (repeating the same test) at least five times. Water content in solvents is analyzed using a Karl-Fischer titrator. Solvent content in water is analyzed with a total organic carbon analyzer or a UV-vis spectrophotometer depending on the GDC used. The bulk NaCl concentration in water is analyzed using a conductivity meter. Individual Na⁺ and Cl⁻ concentrations are measured using a Na⁺ selective electrode and a Cl⁻ selective electrode. The bulk NaCl, Na⁺, and Cl⁻ concentrations of the organic phase are measured using the same methods as above after evaporation of the organic solvent and dissolving the precipitated salt in a known quantity of water (e.g., 10 mL).

Performance evaluate GDCs for brine desalination in TSSE using other salt brines. Effective GDCs are further evaluated using 1.7 mol/L of NaBr, KCl, and KBr to have a consistent molar salt concentration as the NaCl studies to investigate how varying the cation and/or anion affects the equilibrium compositions of the biphasic mixture. All other TSSE testing variables remain the same. Water content in solvents is analyzed using a Karl-Fischer titrator. Solvent content in water is analyzed with a total organic carbon analyzer or a UV-vis spectrophotometer depending on the GDC used. The bulk salt concentrations in water is analyzed using a conductivity meter. Individual Na⁺, K⁺, and Cl⁻ concentrations are measured using a Na⁺ selective electrode, a K⁺ selective electrode, and a Cl⁻ selective electrode. Individual Br concentrations are measured using an ICP-OES. The bulk NaCl, Na⁺, K⁺, Cl⁻, and Br concentrations of the organic phase will be measured using the same methods as above after evaporation of the organic solvent and dissolving the precipitated salt in a known quantity of water (e.g., 10 mL).

Lastly, the GDC(s) that (1) dissolve 10 wt. % water at the lower operating temperature (20-25° C.), (2) dissolve in water at a concentration <500 ppm, (3) have a vapor pressure <10 mm Hg at 80° C. to have minimal solvent evaporative loss, and (4) have a projected toxicity LD₅₀>300 mg/kg bodyweight, are tested with synthetic seawater brine. Synthetic seawater is purchased which conforms to ASTM standard D1141-98. The synthetic seawater is concentrated through a reverse osmosis membrane at ˜50% recovery to produce a synthetic brine to evaluate the GDC(s) for TSSE. All other TSSE testing variables will remain the same. Water content in solvents is analyzed using a Karl-Fischer titrator. Solvent content in water is analyzed with a total organic carbon analyzer or a UV-vis spectrophotometer depending on the GDC used. The bulk salt concentrations in water and the organic phase are analyzed using a conductivity meter. Optionally, individual ion concentrations can be measured using ion selective electrodes or ICP-OES as needed.

The following patents, applications and publications as listed below and throughout this document are hereby incorporated by reference in their entirety herein.

### REFERENCES FOR EXAMPLE 1

(1) Shah, K. M.; Billinge, I. H.; Chen, X.; Fan, H.; Huang, Y.; Winton, R. K.; Yip, N. Y. Drivers, Challenges, and Emerging Technologies for Desalination of High-Salinity Brines: A Critical Review. Desalination 2022, 538, 115827. doi.org/10.1016/j.desal.2022.115827.
(2) Swaminathan, J.; Chung, H. W.; Warsinger, D. M.; Lienhard V, J. H. Energy Efficiency of Membrane Distillation up to High Salinity: Evaluating Critical System Size and Optimal Membrane Thickness. Applied Energy 2018, 211, 715-734. doi.org/10.1016/j.apenergy.2017.11.043.
(3) Thiel, G. P.; Tow, E. W.; Banchik, L. D.; Chung, H. W.; others. Energy Consumption in Desalinating Produced Water from Shale Oil and Gas Extraction. Desalination 2015, 366, 94-112.
(4) Odu, S. O.; van der Ham, A. G. J.; Metz, S.; Kersten, S. R. A. Design of a Process for Supercritical Water Desalination with Zero Liquid Discharge. Ind. Eng. Chem. Res. 2015, 54 (20), 5527-5535. doi.org/10.1021/acs.iecr.5b00826.
(5) Bajpayee, A.; Luo, T.; Muto, A.; Chen, G. Very Low Temperature Membrane-Free Desalination by Directional Solvent Extraction. Energy & Environmental Science 2011, 4 (5), 1672-1675.
(6) Garciadiego, A.; Luo, T.; Dowling, A. W. Molecular Design Targets and Optimization of Low-Temperature Thermal Desalination Systems. Desalination 2021, 504, 114941. doi.org/10.1016/j.desal.2021.114941.
(7) Guo, J.; Luo, S.; Liu, Z.; Luo, T. Direct Arsenic Removal from Water Using Non-Membrane, Low-Temperature Directional Solvent Extraction. Journal of Chemical & Engineering Data 2020, 65 (6), 2938-2946.
(8) Guo, J.; Tucker, Z. D.; Wang, Y.; Ashfeld, B. L.; Luo, T. Ionic Liquid Enables Highly Efficient Low Temperature Desalination by Directional Solvent Extraction. Nature Communications 2021, 12 (1), 1-7.
(9) Davidson, R. R.; Smith Jr, W. H.; Hood, D. W. Structure and Amine-Water Solubility in Desalination by Solvent Extraction. Journal of Chemical and Engineering Data 1960, 5 (4), 420-423.
(10) Boo, C.; Billinge, I. H.; Chen, X.; Shah, K. M.; Yip, N. Y. Zero Liquid Discharge of Ultrahigh-Salinity Brines with Temperature Swing Solvent Extraction. Environmental Science\& Technology 2020, 54 (14), 9124-9131.
(11) Boo, C.; Winton, R. K.; Conway, K. M.; Yip, N. Y. Membrane-Less and Non-Evaporative Desalination of Hypersaline Brines by Temperature Swing Solvent Extraction. Environmental Science \& Technology Letters 2019, 6 (6), 359-364.
(12) Choi, O. K.; Seo, J. H.; Kim, G. S.; Hendren, Z.; Kim, G. D.; Kim, D.; Lee, J. W. Non-Membrane Solvent Extraction Desalination SED Technology Using Solubility-Switchable Amine. Journal of Hazardous Materials 2021, 403, 123636.
(13) Choi, O.; Kim, M.; Cho, A. E.; Choi, Y. C.; Kim, G. D.; Kim, D.; Lee, J. W. Fates of Water and Salts in Non-Aqueous Solvents for Directional Solvent Extraction Desalination: Effects of Chemical Structures of the Solvents. Membrane Water Treatment 2019, 10 (3), 207-212.
(14) Barbosa, G. D.; Dach, E.; Liu, X.; Yip, N. Y.; Turner, C. H. Computational and Experimental Study of Different Brines in Temperature Swing Solvent Extraction Desalination with Amine Solvents. Desalination 2022, 537, 115863. doi.org/10.1016/j.desal.2022.115863.
(15) Ponder, J. W.; Wu, C.; Ren, P.; Pande, V. S.; Chodera, J. D.; Schnieders, M. J.; Haque, I.; Mobley, D. L.; Lambrecht, D. S.; DiStasio, R. A.; Head-Gordon, M.; Clark, G. N. I.; Johnson, M. E.; Head-Gordon, T. Current Status of the AMOEBA Polarizable Force Field. J. Phys. Chem. B 2010, 114 (8), 2549-2564. doi.org/10.1021/jp910674d.
(16) Qi, R.; Jing, Z.; Liu, C.; Piquemal, J.-P.; Dalby, K. N.; Ren, P. Elucidating the Phosphate Binding Mode of Phosphate-Binding Protein: The Critical Effect of Buffer Solution. J. Phys. Chem. B 2018, 122 (24), 6371-6376. doi.org/10.1021/acs.jpcb.8b03194.
(17) Jing, Z.; Liu, C.; Qi, R.; Ren, P. Many-Body Effect Determines the Selectivity for Ca2+ and Mg2+ in Proteins. Proc. Natl. Acad. Sci. U.S.A. 2018, 115 (32). doi.org/10.1073/pnas.1805049115.
(18) Jorgensen, W. L.; Tirado-Rives, J. Potential Energy Functions for Atomic-Level Simulations of Water and Organic and Biomolecular Systems. Proceedings of the National Academy of Sciences 2005, 102 (19), 6665-6670.
(19) Dodda, L. S.; Vilseck, J. Z.; Tirado-Rives, J.; Jorgensen, W. L. 1.14* CM1A-LBCC: Localized Bond-Charge Corrected CM1A Charges for Condensed-Phase Simulations. The Journal of Physical Chemistry B 2017, 121 (15), 3864-3870.
(20) Dodda, L. S.; de Vaca, I.; Tirado-Rives, J.; Jorgensen, W. L. LigParGen Web Server: An Automatic OPLS-AA Parameter Generator for Organic Ligands. Nucleic Acids Research 2017, 45 (W1), W331-W336.
(21) Case, D. A.; Cheatham, T. E.; Darden, T.; Gohlke, H.; Luo, R.; Merz, K. M.; Onufriev, A.; Simmerling, C.; Wang, B.; Woods, R. J. The Amber Biomolecular Simulation Programs. J. Comput. Chem. 2005, 26 (16), 1668-1688. doi.org/10.1002/jcc.20290.
(22) Jakalian, A.; Bush, B. L.; Jack, D. B.; Bayly, C. I. Fast, Efficient Generation of High-Quality Atomic Charges. AM1-BCC Model: I. Method. J. Comput. Chem. 2000, 21 (2), 132-146. doi.org/10.1002/(SICI)1096-987X(20000130)21:2<132::AID-JCC5>3.0.CO;2-P.
(23) Jakalian, A.; Jack, D. B.; Bayly, C. I. Fast, Efficient Generation of High-Quality Atomic Charges. AM1-BCC Model: II. Parameterization and Validation. J. Comput. Chem. 2002, 23 (16), 1623-1641. doi.org/10.1002/jcc.10128.
(24) Davison, R. R. Vapor-Liquid Equilibriums of Water-Diisopropylamine and Water-Di-n-Propylamine. J. Chem. Eng. Data 1968, 13 (3), 348-351. doi.org/10.1021/je60038a013.
(25) Kubat, M. An Introduction to Machine Learning; Springer International Publishing: Cham, 2021. doi.org/10.1007/978-3-030-81935-4.
(26) Rebala, G.; Ravi, A.; Churiwala, S. An Introduction to Machine Learning; Springer International Publishing: Cham, 2019. doi.org/10.1007/978-3-030-15729-6.
(27) Lindahl; Abraham; Hess; Spoel, van der. GROMACS 2021.1 Source Code. 2021. doi.org/10.5281/ZENODO.4561626.
(28) Lindahl; Abraham; Hess; Spoel, van der. GROMACS 2021.1 Manual. 2021. doi.org/10.5281/ZENODO.4561625.
(29) Martinez, L.; Andrade, R.; Birgin, E. G.; Martinez, J. M. PACKMOL: A Package for Building Initial Configurations for Molecular Dynamics Simulations. Journal of Computational Chemistry 2009, 30 (13), 2157-2164.
(30) Nos6, S.; Klein, M. L. Constant Pressure Molecular Dynamics for Molecular Systems. Molecular Physics 1983, 50 (5), 1055-1076.
(31) Parrinello, M.; Rahman, A. Polymorphic Transitions in Single Crystals: A New Molecular Dynamics Method. Journal of Applied Physics 1981, 52 (12), 7182-7190.
(32) Bussi, G.; Donadio, D.; Parrinello, M. Canonical Sampling through Velocity Rescaling. Journal of Chemical Physics 2007, 126 (1), 14101.
(33) Breneman, C. M.; Wiberg, K. B. Determining Atom-Centered Monopoles from Molecular Electrostatic Potentials. The Need for High Sampling Density in Formamide Conformational Analysis. Journal of Computational Chemistry 1990, 11 (3), 361-373.
(34) Frisch, M. J.; Trucks, G. W.; Schlegel, H. B.; Scuseria, G. E.; Robb, M. A.; Cheeseman, J. R.; Montgomery, J. A., Jr.; Vreven, T.; Kudin, K. N.; Burant, J. C.; Millam, J. M.; Iyengar, S. S.; Tomasi, J.; Barone, V.; Mennucci, B.; Cossi, M.; Scalmani, G.; Rega, N.; Petersson, G. A.; Nakatsuji, H.; Hada, M.; Ehara, M.; Toyota, K.; Fukuda, R.; Hasegawa, J.; Ishida, M.; Nakajima, T.; Honda, Y.; Kitao, O.; Nakai, H.; Klene, M.; Li, X.; Knox, J. E.; Hratchian, H. P.; Cross, J. B.; Bakken, V.; Adamo, C.; Jaramillo, J.; Gomperts, R.; Stratmann, R. E.; Yazyev, O.; Austin, A. J.; Cammi, R.; Pomelli, C.; Ochterski, J. W.; Ayala, P. Y.; Morokuma, K.; Voth, G. A.; Salvador, P.; Dannenberg, J. J.; Zakrzewski, V. G.; Dapprich, S.; Daniels, A. D.; Strain, M. C.; Farkas, 0.; Malick, D. K.; Rabuck, A. D.; Raghavachari, K.; Foresman, J. B.; Ortiz, J. V.; Cui, Q.; Baboul, A. G.; Clifford, S.; Cioslowski, J.; Stefanov, B. B.; Liu, G.; Liashenko, A.; Piskorz, P.; Komaromi, I.; Martin, R. L.; Fox, D. J.; Keith, T.; Al-Laham, M. A.; Peng, C. Y.; Nanayakkara, A.; Challacombe, M.; Gill, P. M. W.; Johnson, B.; Chen, W.; Wong, M. W.; Gonzalez, C.; Pople, J. A. Gaussian 09, Revision A.02. Gaussian, Inc. 2009.
(35) Vega, C.; Abascal, J. L. F. Simulating Water with Rigid Non-Polarizable Models: A General Perspective. Phys. Chem. Chem. Phys. 2011, 13 (44), 19663.
(36) Walker, B.; Liu, C.; Wait, E.; Ren, P. Automation of AMOEBA Polarizable Force Field for Small Molecules: Poltype 2. J Comput Chem 2022, 43 (23), 1530-1542.
(37) Klimovich, P. V.; Shirts, M. R.; Mobley, D. L. Guidelines for the Analysis of Free Energy Calculations. Journal of Computer-Aided Molecular Design 2015, 29 (5), 397-411.
(38) Shirts, M. R.; Chodera, J. D. Statistically Optimal Analysis of Samples from Multiple Equilibrium States. Journal of Chemical Physics 2008, 129 (12), 124105.
(39) Shirts, M. R.; Pitera, J. W.; Swope, W. C.; Pande, V. S. Extremely Precise Free Energy Calculations of Amino Acid Side Chain Analogs: Comparison of Common Molecular Mechanics Force Fields for Proteins. Journal of Chemical Physics 2003, 119 (11), 5740-5761.
(40) Bennett, C. H. Efficient Estimation of Free Energy Differences from Monte Carlo Data. Journal of Computational Physics 1976, 22 (2), 245-268.

### REFERENCES FOR EXAMPLE 2

(1) Mekonnen, M. M.; Hoekstra, A. Y. Four billion people facing severe water scarcity. Science Advances 2016, 2 (2), e1500323.
(2) Elimelech, M.; Phillip, W. A. The Future of Seawater Desalination: Energy, Technology, and the Environment. Science 2011, 333 (6043), 712, DOI: 10.1126/science.1200488.
(3) Bajpayee, A.; Luo, T.; Muto, A.; Chen, G. Very low temperature membrane-free desalination by directional solvent extraction. Energy & Environmental Science 2011, 4 (5), 1672-1675, DOI: 10.1039/C1EE01027A.
(4) Boo, C.; Winton, R. K.; Conway, K. M.; Yip, N. Y. Membrane-less and Non-Evaporative Desalination of Hypersaline Brines by Temperature Swing Solvent Extraction. Environmental Science & Technology Letters 2019, 6 (6), 359-364, DOI: 10.1021/acs.estlett.9b00182.
(5) Boo, C.; Billinge, I. H.; Chen, X.; Shah, K. M.; Yip, N. Y. Zero Liquid Discharge of Ultrahigh-Salinity Brines with Temperature Swing Solvent Extraction. Environmental Science & Technology 2020, 54 (14), 9124-9131, DOI: 10.1021/acs.est.0c02555.
(6) Shah, K. M.; Billinge, I. H.; Chen, X.; Fan, H.; Huang, Y.; Winton, R. K.; Yip, N. Y. Drivers, challenges, and emerging technologies for desalination of high-salinity brines: A critical review. Desalination 2022, 538, 115827, DOI: doi.org/10.1016/j.desal.2022.115827.
(7) Johnson, D. T.; Taconi, K. A. The glycerin glut: Options for the value-added conversion of crude glycerol resulting from biodiesel production. Environmental Progress 2007, 26 (4), 338-348, DOI: doi.org/10.1002/ep.10225.
(8) Qian, S.; Liu, X.; Dennis, G. P.; Turner, C. H.; Bara, J. E. Properties of symmetric 1,3-diethers based on glycerol skeletons for CO2 absorption. Fluid Phase Equilibria 2020, 521, 112718, DOI: doi.org/10.1016/j.fluid.2020.112718.
(9) Qian, S.; Liu, X.; Emel'yanenko, V. N.; Sikorski, P.; Kammakakam, I.; Flowers, B. S.; Jones, T. A.; Turner, C. H.; Verevkin, S. P.; Bara, J. E. Synthesis and Properties of 1,2,3-Triethoxypropane: A Glycerol-Derived Green Solvent Candidate. Industrial & Engineering Chemistry Research 2020, 59 (45), 20190-20200, DOI: 10.1021/acs.iecr.0c03789.
(10) Flowers, B. S.; Mittenthal, M. S.; Jenkins, A. H.; Wallace, D. A.; Whitley, J. W.; Dennis, G. P.; Wang, M.; Turner, C. H.; Emel'yanenko, V. N.; Verevkin, S. P.; Bara, J. E. 1,2,3-Trimethoxypropane: A Glycerol-Derived Physical Solvent for CO₂ Absorption. ACS Sustainable Chemistry & Engineering 2017, 5 (1), 911-921, DOI: 10.1021/acssuschemeng.6b02231.
(11) Leal-Duaso, A.; Pdrez, P.; Mayoral, J. A.; Garcia, J. I.; Pires, E. Glycerol-Derived Solvents: Synthesis and Properties of Symmetric Glyceryl Diethers. ACS Sustainable Chemistry & Engineering 2019, 7 (15), 13004-13014, DOI: 10.1021/acssuschemeng.9b02105.
(12) Leal-Duaso, A.; Caballero, M.; Urriolabeitia, A.; Mayoral, J. A.; Garcia, J. I.; Pires, E. Synthesis of 3-alkoxypropan-1,2-diols from glycidol: experimental and theoretical studies for the optimization of the synthesis of glycerol derived solvents. Green Chemistry 2017, 19 (17), 4176-4185, DOI: 10.1039/C7GC01583F.
(13) Sutter, M.; Silva, E. D.; Duguet, N.; Raoul, Y.; Métay, E.; Lemaire, M. Glycerol Ether Synthesis: A Bench Test for Green Chemistry Concepts and Technologies. Chem. Rev. 2015, 115 (16), 8609-8651, DOI: 10.1021/cr5004002.
(14) Sutter, M.; Dayoub, W.; Métay, E.; Raoul, Y.; Lemaire, M. 1,2,3-Trimethoxypropane and Glycerol Ethers as Bio-Sourced Solvents from Glycerol. Synthesis by Solvent-Free Phase-Transfer Catalysis and Utilization as an Alternative Solvent in Chemical Transformations. ChemCatChem 2013, 5 (10), 2893-2904, DOI: 10.1002/cctc.201300458.
(15) Sutter, M.; Pehlivan, L.; Lafon, R.; Dayoub, W.; Raoul, Y.; Metay, E.; Lemaire, M. 1,2,3-Trimethoxypropane, a glycerol-based solvent with low toxicity: new utilization for the reduction of nitrile, nitro, ester, and acid functional groups with TMDS and a metal catalyst. Green Chemistry 2013, 15 (11), 3020-3026, DOI: 10.1039/C3GC41082J.
(16) Huber, G. W.; Iborra, S.; Corma, A. Synthesis of Transportation Fuels from Biomass: Chemistry, Catalysts, and Engineering. Chem. Rev. 2006, 106 (9), 4044-4098, DOI: 10.1021/cr068360d.
(17) Dietrich, P. J.; Lobo-Lapidus, R. J.; Wu, T.; Sumer, A.; Akatay, M. C.; Fingland, B. R.; Guo, N.; Dumesic, J. A.; Marshall, C. L.; Stach, E.; Jellinek, J.; Delgass, W. N.; Ribeiro, F. H.; Miller, J. T. Aqueous Phase Glycerol Reforming by PtMo Bimetallic Nano-Particle Catalyst: Product Selectivity and Structural Characterization. Top. Catal. 2012, 55 (1), 53-69, DOI: 10.1007/s11244-012-9775-5.
(18) Dietrich, P. J.; Akatay, M. C.; Sollberger, F. G.; Stach, E. A.; Miller, J. T.; Delgass, W. N.; Ribeiro, F. H. Effect of Co Loading on the Activity and Selectivity of PtCo Aqueous Phase Reforming Catalysts. ACS Catal. 2014, 4 (2), 480-491, DOI: 10.1021/cs4008705.
(19) Pan, Y.; Wu, G.; He, Y.; Feng, J.; Li, D. Identification of the Au/ZnO interface as the specific active site for the selective oxidation of the secondary alcohol group in glycerol. J. Catal. 2019, 369, 222-232, DOI: 10.1016/j.jcat.2018.10.030.
(20) Rahmat, N.; Abdullah, A. Z.; Mohamed, A. R. Recent progress on innovative and potential technologies for glycerol transformation into fuel additives: A critical review. Renewable and Sustainable Energy Reviews 2010, 14 (3), 987-1000, DOI: 10.1016/j.rser.2009.11.010.
(21) Agency, I. E. Bioenergy—Fuels & Technologies. iea.org/fuels-and-technologies/bioenergy (accessed 9 Feb. 2021).
(22) Almena, A.; Martin, M. Technoeconomic Analysis of the Production of Epichlorohydrin from Glycerol. Industrial & Engineering Chemistry Research 2016, 55 (12), 3226-3238, DOI: 10.1021/acs.iecr.5b02555.
(23) Santacesaria, E.; Tesser, R.; Di Serio, M.; Casale, L.; Verde, D. New Process for Producing Epichlorohydrin via Glycerol Chlorination. Industrial & Engineering Chemistry Research 2010, 49 (3), 964-970, DOI: 10.1021/ie900650x.
(24) Davison, R.; Smith Jr, W.; Hood, D. W. Structure and Amine-Water Solubility in Desalination by Solvent Extraction. Journal of Chemical and Engineering Data 1960, 5 (4), 420-423.
(25) Luo, T.; Bajpayee, A.; Chen, G. Directional solvent for membrane-free water desalination-A molecular level study. Journal of Applied Physics 2011, 110 (5), 054905, DOI: 10.1063/1.3627239.
(26) Rish, D.; Luo, S.; Kurtz, B.; Luo, T. Exceptional ion rejection ability of directional solvent for non-membrane desalination. Applied Physics Letters 2014, 104 (2), 024102, DOI: 10.1063/1.4861835.
(27) Sanap, D. B.; Kadam, K. D.; Narayan, M.; Kasthurirangan, S.; Nemade, P. R.; Dalvi, V. H. Analysis of saline water desalination by directed solvent extraction using octanoic acid. Desalination 2015, 357, 150-162, DOI: doi.org/10.1016/j.desal.2014.11.020.
(28) Alotaibi, S.; Ibrahim, O. M.; Luo, S.; Luo, T. Modeling of a continuous water desalination process using directional solvent extraction. Desalination 2017, 420, 114-124, DOI: doi.org/10.1016/j.desal.2017.07.004.
(29) Choi, O. K.; Seo, J. H.; Kim, G. S.; Hendren, Z.; Kim, G. D.; Kim, D.; Lee, J. W. Non-membrane solvent extraction desalination (SED) technology using solubility-switchable amine. Journal of Hazardous Materials 2021, 403, 123636, DOI: doi.org/10.1016/j.jhazmat.2020.123636.
(30) McNally, J. S.; Foo, Z. H.; Deshmukh, A.; Orme, C. J.; Lienhard, J. H.; Wilson, A. D. Solute displacement in the aqueous phase of water-NaCl-organic ternary mixtures relevant to solvent-driven water treatment. RSC Advances 2020, 10 (49), 29516-29527, DOI: 10.1039/DORA06361D.
(31) Deshmukh, A.; Foo, Z. H.; Stetson, C.; Lee, H.; Orme, C. J.; Wilson, A. D.; Lienhard, J. H. Thermodynamics of solvent-driven water extraction from hypersaline brines using dimethyl ether. Chemical Engineering Journal 2022, 434, 134391, DOI: doi.org/10.1016/j.cej.2021.134391.
(32) Garciadiego, A.; Luo, T.; Dowling, A. W. Molecular design targets and optimization of low-temperature thermal desalination systems. Desalination 2021, 504, 114941, DOI: doi.org/10.1016/j.desal.2021.114941.
(33) Guo, J.; Tucker, Z. D.; Wang, Y.; Ashfeld, B. L.; Luo, T. Ionic liquid enables highly efficient low temperature desalination by directional solvent extraction. Nature Communications 2021, 12 (1), 437, DOI: 10.1038/s41467-020-20706-y.
(34) Qian, S.; Ward, L. M.; Rakers, L. S.; Weinman, S. T.; Bara, J. E. 1-Propyl-4(5)-Methylimidazole Isomers for Temperature Swing Solvent Extraction. Molecules 2022, 27 (17), 5583.
(35) Chandran, P.; Shah, J. K. A molecular simulation approach to the computation of mutual solubility of water and organic liquids: Application to fatty acids. Fluid Phase Equilibria 2018, 472, 48-55, DOI: doi.org/10.1016/j.fluid.2018.05.002.
(36) Kim, M.; Choi, O. K.; Cho, Y.; Lee, J. W.; Cho, A. E. Elucidation of the desalination mechanism of solvent extraction method through molecular modeling studies. Desalination 2020, 496, 114704, DOI: doi.org/10.1016/j.desal.2020.114704.
(37) Choi, O.; Kim, M.; Cho, A. E.; Choi, Y. C.; Kim, G. D.; Kim, D.; Lee, J. W. Fates of water and salts in non-aqueous solvents for directional solvent extraction desalination: Effects of chemical structures of the solvents. Membrane Water Treatment 2019, 10 (3), 207-212.
(38) Barbosa, G. D.; Bara, J. E.; Weinman, S. T.; Turner, C. H. Molecular aspects of temperature swing solvent extraction for brine desalination using imidazole-based solvents. Chemical Engineering Science 2022, 247, 116866, DOI: doi.org/10.1016/j.ces.2021.116866.
(39) Sappidi, P.; Barbosa, G.; Rabideau, B. D.; Weinman, S. T.; Turner, C. H. Molecular Simulation of High-Salinity Brines in Contact with Diisopropylamine and Tripropylamine Solvents. Industrial & Engineering Chemistry Research 2021, 60 (21), 7917-7925, DOI: 10.1021/acs.iecr.1c01057.
(40) Barbosa, G. D.; Liu, X.; Bara, J. E.; Weinman, S. T.; Turner, C. H. High-salinity brine desalination with amine-based temperature swing solvent extraction: A molecular dynamics study. Journal of Molecular Liquids 2021, 341, 117359, DOI: doi.org/10.1016/j.molliq.2021.117359.
(41) Liu, X.; Turner, C. Electronic structure calculations of the fundamental interactions in solvent extraction desalination. Journal of Molecular Liquids 2022, 364, 119986, DOI: doi.org/10.1016/j.molliq.2022.119986.
(42) Barbosa, G. D.; Dach, E.; Liu, X.; Yip, N. Y.; Turner, C. H. Computational and experimental study of different brines in temperature swing solvent extraction desalination with amine solvents. Desalination 2022, 537, 115863, DOI: doi.org/10.1016/j.desal.2022.115863.
(43) Alder, C. M.; Hayler, J. D.; Henderson, R. K.; Redman, A. M.; Shukla, L.; Shuster, L. E.; Sneddon, H. F. Updating and further expanding GSK's solvent sustainability guide. Green Chemistry 2016, 18 (13), 3879-3890, DOI: 10.1039/C6GC00611F.
(44) Garcia, J. I.; Pires, E.; Aldea, L.; Lomba, L.; Perales, E.; Giner, B. Ecotoxicity studies of glycerol ethers in Vibrio fischeri: checking the environmental impact of glycerol-derived solvents. Green Chemistry 2015, 17 (8), 4326-4333, DOI: 10.1039/C5GC00857C.
(45) Garcia, J. I.; Garcia-Marin, H.; Mayoral, J. A.; Pérez, P. Green solvents from glycerol. Synthesis and physico-chemical properties of alkyl glycerol ethers. Green Chemistry 2010, 12 (3), 426-434, DOI: 10.1039/B923631G.
(46) Shannon, M. S.; Tedstone, J. M.; Danielsen, S. P. O.; Bara, J. E. Evaluation of Alkylimidazoles as Physical Solvents for C02/CH4 Separation. Industrial & Engineering Chemistry Research 2012, 51 (1), 515-522, DOI: 10.1021/ie202111k.
(47) Shannon, M. S.; Bara, J. E. Reactive and Reversible Ionic Liquids for CO2 Capture and Acid Gas Removal. Separation Science and Technology 2012, 47 (2), 178-188, DOI: 10.1080/01496395.2011.630055.
(48) Shannon, M. S.; Bara, J. E. Properties of Alkylimidazoles as Solvents for CO2 Capture and Comparisons to Imidazolium-Based Ionic Liquids. Industrial & Engineering Chemistry Research 2011, 50 (14), 8665-8677, DOI: 10.1021/ie200259h.
(49) Bara, J. E.; Carlisle, T. K.; Gabriel, C. J.; Camper, D.; Finotello, A.; Gin, D. L.; Noble, R. D. Guide to CO2 Separations in Imidazolium-Based Room-Temperature Ionic Liquids. Industrial & Engineering Chemistry Research 2009, 48 (6), 2739-2751, DOI: 10.1021/ie8016237.
(50) Wudarczyk, J.; Papamokos, G.; Margaritis, V.; Schollmeyer, D.; Hinkel, F.; Baumgarten, M.; Floudas, G.; Mullen, K. Hexasubstituted Benzenes with Ultrastrong Dipole Moments. Angewandte Chemie International Edition 2016, 55 (9), 3220-3223, DOI: 10.1002/anie.201508249.
(51) Barbosa, G. D.; Bara, J. E.; Turner, C. H. Molecular simulation of glycerol-derived triether podands for lithium ion solvation. Phys. Chem. Chem. Phys. 2022, 24 (16), 9459-9466, DOI: 10.1039/D2CP00646D.
(52) Barbosa, G. D.; Turner, C. H. Molecular-level solvation and selectivity behavior of Na+, K+, and Li+ within glycerol-derived solvents. Chem. Eng. Sci. 2022, 262, 117992, DOI: doi.org/10.1016/j.ces.2022.117992.
(53) Arthur, T.; Harjani, J. R.; Phan, L.; Jessop, P. G.; Hodson, P. V. Effects-driven chemical design: the acute toxicity of C02-triggered switchable surfactants to rainbow trout can be predicted from octanol-water partition coefficients. Green Chemistry 2012, 14 (2), 357-362, DOI: 10.1039/C1GC15620A.
(54) Liu, H.; Bara, J. E.; Turner, C. H. DFT study on the effect of exocyclic substituents on the proton affinity of 1-methylimidazole. Chemical Physics 2013, 416, 21-25, DOI: doi.org/10.1016/j.chemphys.2013.02.029.
(55) Lu, T., Molclus Program, Version 1.9.5. Beijing Kein Research Center for Natural Science, China. keinsci.com/research/molclus.html (accessed Jul. 1, 2020): 2016.
(56) Pracht, P.; Bohle, F.; Grimme, S. Automated Exploration of the Low-Energy Chemical Space with Fast Quantum Chemical Methods. Phys. Chem. Chem. Phys. 2020, 22, 7169-7192.
(57) Grimme, S.; Bannwarth, C.; Shushkov, P. A Robust and Accurate Tight-Binding Quantum Chemical Method for Structures, Vibrational Frequencies, and Noncovalent Interactions of Large Molecular Systems Parametrized for All spd-Block Elements (Z=1-86). J. Chem. Theory Comput. 2017, 13, 1989-2009.
(58) Pracht, P.; Caldeweyher, E.; Ehlert, S.; Grimme, S. A Robust Non-Self-Consistent Tight-Binding Quantum Chemistry Method for large Molecules. ChemRxiv 2019, preprint (DOI: 10.26434/chemrxiv.8326202.v1).
(59) Bannwarth, C.; Ehlert, S.; Grimme, S. GFN2-xTB—An Accurate and Broadly Parametrized Self-Consistent Tight-Binding Quantum Chemical Method with Multipole Electrostatics and Density-Dependent Dispersion Contributions. J. Chem. Theory Comput. 2019, 15, 1652-1671.
(60) Brandenburg, J. G.; Bannwarth, C.; Hansen, A.; Grimme, S. B97-3c: A Revised Low-Cost Variant of the B97-D Density Functional Method. J. Chem. Phys. 2018, 148, 064104.
(61) Neese, F. The ORCA Program System. Wiley Interdiscip. Rev.: Comput. Mol. Sci. 2012, 2, 73-78.
(62) Neese, F. Software Update: the ORCA Program System, Version 4.0. Wiley Interdiscip. Rev.: Comput. Mol. Sci. 2018, 8, e1327.
(63) Abedini, A.; Crabtree, E.; Bara, J. E.; Turner, C. H. Molecular Simulation of Ionic Polyimides and Composites with Ionic Liquids as Gas-Separation Membranes. Langmuir 2017, 33 (42), 11377-11389, DOI: 10.1021/acs.langmuir.7b01977.
(64) Shannon, M. S.; Irvin, A. C.; Liu, H.; Moon, J. D.; Hindman, M. S.; Turner, C. H.; Bara, J. E. Chemical and Physical Absorption of SO₂ by N-Functionalized Imidazoles: Experimental Results and Molecular-level Insight. Industrial & Engineering Chemistry Research 2015, 54 (1), 462-471, DOI: 10.1021/ie503752h.
(65) Liu, H.; Zhang, Z.; Bara, J. E.; Turner, C. H. Electrostatic Potential within the Free Volume Space of Imidazole-Based Solvents: Insights into Gas Absorption Selectivity. Journal of Physical Chemistry B 2014, 118 (1), 255-264, DOI: 10.1021/jp410143j.
(66) Qian, S.; Liu, X.; Turner, C. H.; Bara, J. E. Synthesis and properties of symmetric glycerol-derived 1,2,3-triethers and 1,3-diether-2-ketones for CO2 absorption. Chem. Eng. Sci. 2022, 248, 117150, DOI: doi.org/10.1016/j.ces.2021.117150.
(67) Qian, S.; Liu, X.; Turner, C. H.; Bara, J. E. Glycerol-derived solvents containing two or three distinct functional groups enabled by trifluoroethyl glycidyl ether. AIChE Journal 2022, 68 (3), e17533, DOI: doi.org/10.1002/aic.17533.
(68) Andreeva, I. V.; Zaitsau, D. H.; Qian, S.; Turovtzev, V. V.; Pimerzin, A. A.; Bara, J. E.; Verevkin, S. P. Glycerol valorisation towards biofuel additivities: Thermodynamic studies of glycerol ethers. Chem. Eng. Sci. 2022, 247, 117032, DOI: doi.org/10.1016/j.ces.2021.117032.
(69) U.S. Pat. No. 8,501,007
(71) U.S. Pat. No. 9,428,404
(72) US 2023/0061678A1
(73) US 20230174391A9
(74) WO 2022/034353A1
(75) WO 2023/220329A1
(76) U.S. Pat. No. 11,845,038
(77) Ward, Lauren M., et al. “Substituent Effects on the Desalination Performance of Six-Carbon Amines in Temperature Swing Solvent Extraction.” Industrial & Engineering Chemistry Research (2025).

What is claimed is:

1. A method for desalinating high-salinity brines, the method comprising:
contacting a feed brine with an amine-based solvent under conditions effective for temperature swing solvent extraction (TSSE) to produce a substantially desalinated product,
wherein the amine-based solvent comprises sec-butyl-isopropyl-amine and/or a glycerol-based amine solvent.

2. The method of claim 1, wherein the glycerol-based amine solvent is represented by Formula I, or a salt thereof: 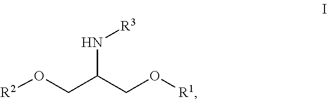
wherein each R¹, R², and R³ is independently hydrogen, a substituted or unsubstituted linear, branch, or cyclic alkyl, a substituted or unsubstituted linear, branch, or cyclic alkoxy, a substituted or unsubstituted linear, branch, or cyclic alkoxyalkyl, or a substituted or unsubstituted linear, branch, or cyclic alkenyl;
with the proviso that at least one of R¹, R², and R³ is not hydrogen.

3. The method of claim 2, wherein each R¹, R², and R³ is independently hydrogen, a substituted or unsubstituted linear, branch, or cyclic C₁-C₆ alkyl, a substituted or unsubstituted linear, branch, or cyclic C₁-C₆ alkoxy, a substituted or unsubstituted linear, branch, or cyclic Cl₁- C₆ alkoxyalkyl, or a substituted or unsubstituted linear, branch, or cyclic C₁-C₆ alkenyl;
with the proviso that at least one of R¹, R², and R³ is not hydrogen.

4. The method of claim 2, wherein each R¹, R², and R³ is independently hydrogen, a substituted or unsubstituted linear, branch, or cyclic C₁-C₃ alkyl, a substituted or unsubstituted linear, branch, or cyclic C₁-C₃ alkoxy, a substituted or unsubstituted linear, branch, or cyclic C₁-C₃ alkoxyalkyl, or a substituted or unsubstituted linear, branch, or cyclic C₁-C₆ alkenyl;
with the proviso that at least one of R¹, R², and R³ is not hydrogen.

5. The method of claim 2, wherein R¹ and R² are the same.

6. The method of claim 2, wherein R¹ and R² are different.

7. The method of claim 1, wherein the glycerol-based amine solvent is represented by Formula II, or a salt thereof: 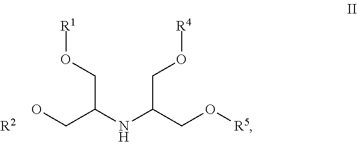
wherein each R¹, R², R⁴, and R⁵ is independently hydrogen, a substituted or unsubstituted linear, branch, or cyclic alkyl, a substituted or unsubstituted linear, branch, or cyclic alkoxy, a substituted or unsubstituted linear, branch, or cyclic alkoxyalkyl, or a substituted or unsubstituted linear, branch, or cyclic alkenyl;
with the proviso that at least one of R¹, R², R⁴, and R⁵ is not hydrogen.

8. The method of claim 7, wherein each R¹, R², R⁴, and R⁵ is independently hydrogen, a substituted or unsubstituted linear, branch, or cyclic C₁-C₆ alkyl, a substituted or unsubstituted linear, branch, or cyclic C₁-C₆ alkoxy, a substituted or unsubstituted linear, branch, or cyclic C₁-C₆alkoxyalkyl, or a substituted or unsubstituted linear, branch, or cyclic C₁-C₆ alkenyl;
with the proviso that at least one of R¹, R², R⁴, and R⁵ is not hydrogen.

9. The method of claim 7, wherein each R¹, R², R⁴, and R⁵ is independently hydrogen, a substituted or unsubstituted linear, branch, or cyclic C₁-C₃ alkyl, a substituted or unsubstituted linear, branch, or cyclic C₁-C₃ alkoxy, a substituted or unsubstituted linear, branch, or cyclic C₁-C₃ alkoxyalkyl, or a substituted or unsubstituted linear, branch, or cyclic C₁-C₆ alkenyl;
with the proviso that at least one of R¹, R², R⁴, and R⁵ is not hydrogen.

10. The method of claim 7, wherein R¹, R², R⁴, and R⁵ are the same.

11. The method of claim 1, wherein the amine-based solvent comprises more than one amine-based solvent.

12. The method of claim 1, wherein the conditions effective for TSSE comprise:
contacting the feed brine with the amine-based solvent at a first temperature to form an extractant phase comprising water from the feed brine dissolved in the solvent and a raffinate;
separating the extractant phase and the raffinate;
heating or cooling the extractant phase to second temperature to thereby adjust the miscibility of the water in the amine-based solved to form the substantially desalinated water product and a solvent phase; and
separating the substantially desalinated water product from the solvent phase.

13. The method of claim 12, further comprising recycling the separated solvent phase for additional TSSE processes.

14. The method of claim 1, wherein the feed brine has a total dissolved solids of 50,000 ppm or more.

15. A system for desalinating high-salinity brines, the system comprising:
an extraction unit configured to receive a feed brine and an amine-based solvent and contact the feed brine and amine-based solvent under conditions effective for temperature swing solvent extraction (TSSE) to produce a substantially desalinated product,
wherein the amine-based solvent comprises sec-butyl-isopropyl-amine and/or a glycerol-based amine solvent.
